(19) United States
(12) Patent Application Publication
(10) Pub. No.: US 2015/0175650 A1
(43) Pub. Date: Jun. 25, 2015

(54) SYNTHESIS OF ENT-PROGESTERONE AND INTERMEDIATES THEREOF

(71) Applicant: Prevacus, Inc., Tailahassee, FL (US)

(72) Inventors: Daniel E. Levy, San Mateo, CA (US); Faliang Zhang, Beijing (CN); Xinxi Zhan, Beijing (CN)

(21) Appl. No.: 14/573,656

(22) Filed: Dec. 17, 2014

Related U.S. Application Data
(60) Provisional application No. 61/919,420, filed on Dec. 20, 2013.

(51) Int. Cl. C07J 1/00; C07J 13/00; C07J 75/00; C07D 317/26; C07D 317/72; C07D 317/30

(52) U.S. Cl. CPC C07J 1/00; C07D 317/72; C07D 317/30; C07J 75/005; C07D 317/26; C07J 13/007

(57) ABSTRACT

The present invention relates to the synthesis of ent-progesterone and intermediates thereof.

### RELATED U.S. APPLICATION DATA

U.S. Provisional Application No. 61/919,420, filed on Dec. 20, 2013, which is incorporated herein by reference in its entirety.

### FIELD OF THE INVENTION

The present invention relates to the synthesis of ent-progesterone and intermediates thereof.

### BACKGROUND

Progesterone is a C-21 steroid hormone involved in the female menstrual cycle, pregnancy and embryogenesis of humans and other species. Progesterone belongs to a class of hormones called progestogens, and is the major naturally occurring human progestogen.

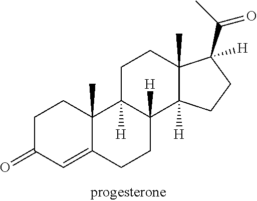

Progesterone is naturally produced by the ovaries of mammals, but can also be produced by some plants and yeast. An economical semi-synthesis of progesterone from the plant steroid diosgenin isolated from yams was developed by Russell Marker in 1940 for the Parke-Davis pharmaceutical company [Marker R E, Krueger J (1940). “Sterols. CXII. Sapogenins. XLI. The Preparation of Trillin and its Conversion to Progesterone”. J. Am. Chem. Soc. 62 (12): 3349-3350]. This synthesis is known as the Marker degradation. Additional semi-syntheses of progesterone have also been reported starting from a variety of steroids. For the example, cortisone can be simultaneously deoxygenated at the C-17 and C-21 position by treatment with iodotrimethylsilane in chloroform to produce 11-keto-progesterone (ketogestin), which in turn can be reduced at position-11 to yield progesterone. [Numazawa M, Nagaoka M, Kunitama Y (September 1986). “Regiospecific deoxygenation of the dihydroxyacetone moiety at C-17 of corticoid steroids with iodotrimethylsilane”. Chem. Pharm. Bull. 34 (9): 3722-6].

A total synthesis of progesterone was reported in 1971 by W. S. Johnson. [Johnson W S, Gravestock M B, McCarry B E (August 1971). “Acetylenic bond participation in biogenetic-like olefinic cyclizations. II. Synthesis of dl-progesterone”. J. Am. Chem. Soc. 93 (17): 4332-4].

The use of progesterone and its analogues have many medical applications, both to address acute situations and to address the long-term decline of natural progesterone levels. Other uses of progesterone include the prevention of preterm birth, to control anovulatury bleeding, to increase skin elasticity and bone strength, and to treat multiple sclerosis.

Progesterone is also useful for the treatment of traumatic brain injury: it reduces poor outcomes following injury by inhibiting inflammatory factors (TNF-α and IL-1β) and subsequently reducing brain edema (Pan, D., et al. (2007), Biomed Environ Sci 20, 432-438; Jiang, C., et al. (2009), Inflamm Res 58, 619-624.) Prog-treated rats have demonstrated significant improvements on a Neurological Severity Score (test for motor and cognitive functioning) following injury (Roof, R. L., et al. (1992), Restor Neurol Neurosci 4, 425-427). Administering Prog or its derivative allopregnanolone (ALLO) also results in a decrease of the presence of the factors of cell death (caspase-3) and gliosis (GFAP) (Cutler, S. M., et al. (2007), J Neurotrauma 24, 1475-1486) following injury (VanLandingham, J. W., et al. (2007), Neurosci Lett 425, 94-98; Wright, D. W., et al. (2007), Ann Emerg Med 49, 391-402, 402 e391-392). See also, Progesterone for the Treatment of Traumatic Brain Injury (ProTECT III), ClinicalTrials.gov Identifier:NCT00822900; Efficacy and Safety Study of Intravenous Progesterone in Patients With Severe Traumatic Brain Injury (SyNAPSe), ClinicalTrials.gov Identifier: NCT01143064; Progesterone Treatment of Blunt Traumatic Brain Injury, ClinicalTrials.gov Identifier: NCT00048646; and Blood Tests to Study Injury Severity and Outcome in Traumatic Brain Injury Patients (BioProTECT), ClinicalTrials.gov Identifier: NCT01730443. See further, ProTEC™III, Progesterone for the Treatment of Traumatic Brain Injury; Progesterone for Traumatic Brain Injury Tested in Phase III Clinical Trial; BHR Pharma Investigational Traumatic Brain Injury Treatment Receives European Medicines Agency Orphan Medicinal Product Designation; and BHR Pharma SyNAPSe® Trial DSMB Data Analyses Determine No Safety Issues; Study Should Continue to Conclusion at http://www.pmewswire.com/news-releases/bhr-pharma-synapse-trial-dsmb-data-analyses-determine-no-safety-issues-study-should-continue-to-conclusion-187277871.html.

Progesterone exists in a non-naturally occurring enantiomeric form known as ent-progesterone.

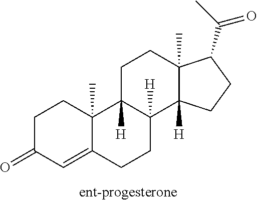

ent-Progesterone has been shown to have equal efficacy to natural progesterone in reducing cell death, brain swelling, and inflammation while the enantiomer has three times the antioxidant activity of racemate. Similarly, ent-progesterone has been found to have fewer sexual side effects such as suppression of spermatogenesis; inhibition of the conversion of testosterone to dihydrotestosterone; reduction in the size of the testes, epididymis, and leydig cells; and no hyper-coagulative risk as may be seen with natural progesterone. In addition, utilities for ent-progesterone have been described in U.S. patent application Ser. No. 13/645,881, which was filed on Oct. 5, 2012 and is entitled “Nasal Delivery Mechanism for Prophylatic and Post-Acute Use for Progesterone and/or Its Enantiomer for Use in Treatment of Mild Traumatic Brain Injuries, U.S. patent application Ser. No. 13/645,854, which was filed on Oct. 12, 2012 and is entitled “Prophylactic and Post-Acute Use of Progesterone and Its Enantiomer to Better Outcomes Associated with Concussion,” and U.S. patent application Ser. No. 13/645,925, which was filed on Oct. 12, 2012 and is entitled “Prophylactic and Post-15 Acute Use of Progesterone in Conjunction with Its Enantiomer for Use in Treatment of Traumatic Brain Injuries, the entire contents and disclosures each of which are incorporated herein by reference in their entireties. See also VanLandingham et al., Neuropharmacology, The enantiomer of progesterone acts as a molecular neuroprotectant after traumatic brain injury, 2006, 51, 1078-1085.

Nevertheless, previous attempts to synthesize ent-progesterone have been difficult and suffers from poor yields; use of hazardous reagents and conditions; and numerous and costly reaction steps making the commercial use and scale-up of ent-progesterone unfeasible.

As such, there exists a need for an efficient synthesis of ent-progesterone.

### SUMMARY OF THE INVENTION

In one aspect, the invention provides a method for preparing ent-progesterone comprising reacting a compound of the formula:

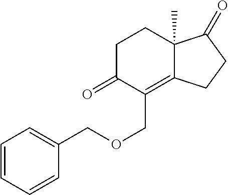 [SMILES: C[C@@]12CCC(=O)C(COCc3ccccc3)=C1CCC2=O]

to produce a compound of the formula:

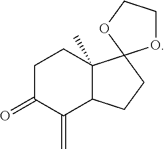 [SMILES: C=C1C(=O)CC[C@]2(C)C1CCC21OCCO1]

In another aspect, the invention provides a method for preparing ent-progesterone comprising reacting a compound of the formula:

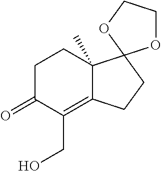 [SMILES: C[C@@]12CCC(=O)C(CO)=C1CCC21OCCO1]

to produce a compound of the formula:

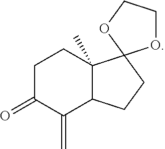 [SMILES: C=C1C(=O)CC[C@]2(C)C1CCC21OCCO1]

In certain embodiments, the compound of the formula:

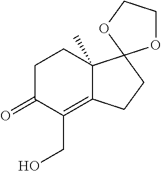 [SMILES: C[C@@]12CCC(=O)C(CO)=C1CCC21OCCO1]

is prepared by subjecting a compound of the formula:

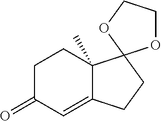

to a Baylis-Hillman reaction.

In still another aspect, the invention provides a method for preparing ent-progesterone comprising reacting a compound of the formula:

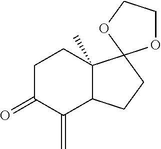 [SMILES: C=C1C(=O)CC[C@]2(C)C1CCC21OCCO1]

with a compound of the formula:

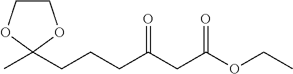 [SMILES: CCOC(=O)CC(=O)CCCC1(C)OCCO1]

to produce a compound of the formula:

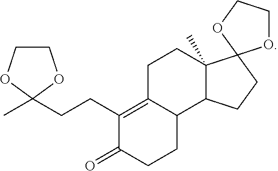 [SMILES: CC1(CCC2=C3CC[C@]4(C)C(CCC45OCCO5)C3CCC2=O)OCCO1]

In certain embodiments, the compound of the formula

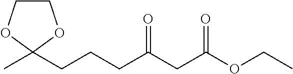 [SMILES: CCOC(=O)CC(=O)CCCC1(C)OCCO1]

is prepared by reacting a compound of the formula:

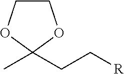

wherein R is any leaving group
with a compound of the formula:

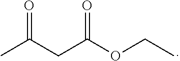

In certain embodiments, and without being limited thereto, leaving group R is —OTs, —OMs, —OTf, —Cl, —Br, or —I. In still other embodiments, leaving group R is —OTs, —Br, or —I. In yet other embodiments, leaving group R is —Br.

In another aspect, the invention provides a method for preparing ent-progesterone comprising reacting a compound of the formula:

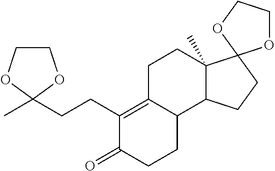 [SMILES: CC1(CCC2=C3CC[C@]4(C)C(CCC45OCCO5)C3CCC2=O)OCCO1]

to produce a compound of the formula:

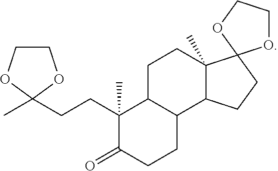 [SMILES: CC1(CC[C@]2(C)C(=O)CCC3C2CC[C@]2(C)C3CCC23OCCO3)OCCO1]

In certain embodiments, the compound of the formula:

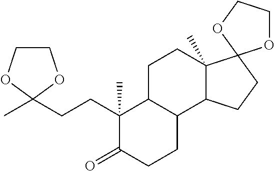 [SMILES: CC1(CC[C@]2(C)C(=O)CCC3C2CC[C@]2(C)C3CCC23OCCO3)OCCO1]

is prepared by subjecting a compound of the formula:

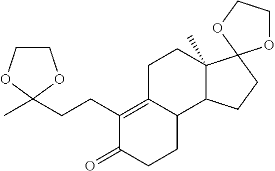 [SMILES: CC1(CCC2=C3CC[C@]4(C)C(CCC45OCCO5)C3CCC2=O)OCCO1]

to a Birch-type reduction followed by methylation.

In certain embodiments, the compound of the formula:

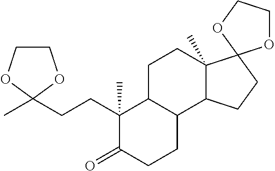 [SMILES: CC1(CC[C@]2(C)C(=O)CCC3C2CC[C@]2(C)C3CCC23OCCO3)OCCO1]

is prepared by subjecting a compound of the formula:

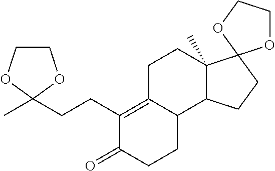 [SMILES: CC1(CCC2=C3CC[C@]4(C)C(CCC45OCCO5)C3CCC2=O)OCCO1]

to a reductive silylation reaction followed by de-silylation and methylation.

In still another aspect, the invention provides a method for preparing ent-progesterone comprising reacting a compound of the formula:

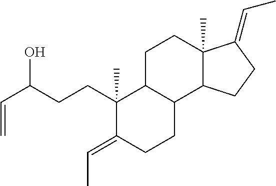 [SMILES: C=CC(O)CC[C@]1(C)/C(=C/C)CCC2C3CC/C(=C\C)[C@]3(C)CCC21]

to produce a compound of the formula:

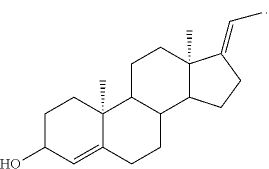 [SMILES: C/C=C1\CCC2C3CCC4=CC(O)CC[C@@]4(C)C3CC[C@@]12C]

In yet another aspect, the invention provides a method for preparing ent-progesterone comprising reacting a compound of the formula:

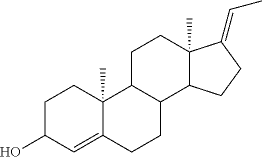 [SMILES: C/C=C1\CCC2C3CCC4=CC(O)CC[C@@]4(C)C3CC[C@@]12C]

to produce a compound of the formula:

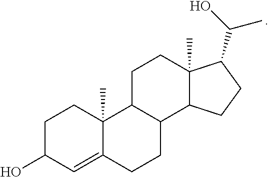 [SMILES: CC(O)[C@@H]1CCC2C3CCC4=CC(O)CC[C@@]4(C)C3CC[C@]21C]

In still yet another aspect, the invention provides a method for preparing ent-progesterone comprising reacting a compound of the formula:

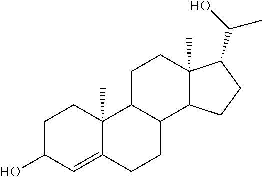 [SMILES: CC(O)[C@@H]1CCC2C3CCC4=CC(O)CC[C@@]4(C)C3CC[C@]21C]

to produce a compound of the formula:

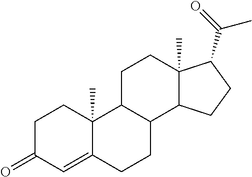 [SMILES: CC(=O)[C@@H]1CCC2C3CCC4=CC(=O)CC[C@@]4(C)C3CC[C@]21C]

(ent-Progesterone).

In one aspect, the invention provides a method for preparing ent-progesterone comprising reacting a compound of the formula:

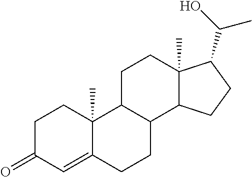 [SMILES: CC(O)[C@@H]1CCC2C3CCC4=CC(=O)CC[C@@]4(C)C3CC[C@]21C]

to produce a compound of the formula:

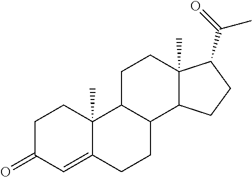 [SMILES: CC(=O)[C@@H]1CCC2C3CCC4=CC(=O)CC[C@@]4(C)C3CC[C@]21C]

(ent-Progesterone).

In another aspect, the invention provides a method for preparing ent-progesterone comprising reacting a compound of the formula:

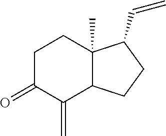

with a compound of the formula

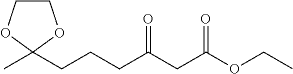 [SMILES: CCOC(=O)CC(=O)CCCC1(C)OCCO1]

to produce a compound of the formula

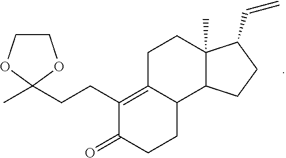 [SMILES: C=C[C@@H]1CCC2C3CCC(=O)C(CCC4(C)OCCO4)=C3CC[C@]21C]

In certain embodiments, the compound of the formula

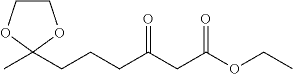 [SMILES: CCOC(=O)CC(=O)CCCC1(C)OCCO1]

is prepared by reacting a compound of the formula:

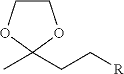

wherein R is any leaving group
with a compound of the formula:

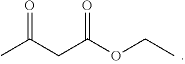

In certain embodiments, and without being limited thereto, leaving group R is —OTs, —OMs, —OTf, —Cl, —Br, or —I. In still other embodiments, leaving group R is —OTs, —Br, or —I. In yet other embodiments, leaving group R is —Br.

In another aspect, the invention provides a method for preparing ent-progesterone comprising the step of reacting a compound of the formula:

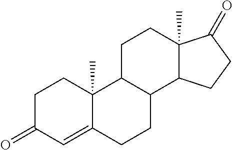 [SMILES: C[C@@]12CCC3C(CCC4=CC(=O)CC[C@]43C)C1CCC2=O]

to produce a compound of the formula

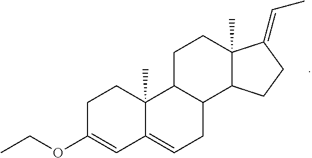 [SMILES: C/C=C1\CCC2C3CC=C4C=C(OCC)CC[C@@]4(C)C3CC[C@@]12C]

In still another aspect, the invention provides a method for preparing ent-progesterone comprising the step of reacting a compound of the formula:

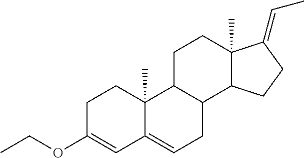 [SMILES: C/C=C1\CCC2C3CC=C4C=C(OCC)CC[C@@]4(C)C3CC[C@@]12C]

to produce a compound of the formula

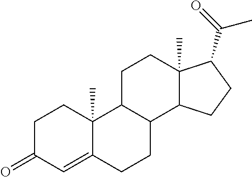 [SMILES: CC(=O)[C@@H]1CCC2C3CCC4=CC(=O)CC[C@@]4(C)C3CC[C@]21C]

(ent-Progesterone).

In yet another aspect, the invention provides a method for preparing ent-progesterone comprising the step of reacting a compound of the formula:

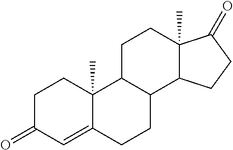 [SMILES: C[C@@]12CCC3C(CCC4=CC(=O)CC[C@]43C)C1CCC2=O]

to produce a compound of the formula

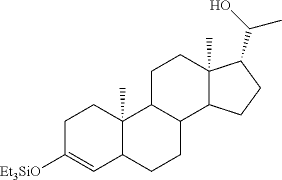

via reductive silylation.

In still another aspect, the Invention provides a method for preparing ent-progesterone comprising the step of reacting a compound of the formula:

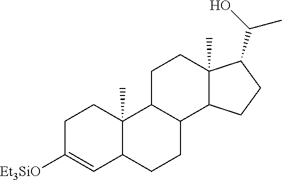

to produce a compound of the formula

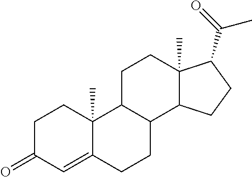 [SMILES: CC(=O)[C@@H]1CCC2C3CCC4=CC(=O)CC[C@@]4(C)C3CC[C@]21C]

(ent-Progesterone).

In another aspect, the invention provides a method for preparing ent-progesterone comprising the step of reacting an enone intermediate compound with triethylsilane and a catalyst to form a silyl enol ether.

In certain embodiments, the invention provides a method for preparing ent-progesterone comprising two or more of the steps described above. In other embodiments, the invention provides a method for preparing ent-progesterone comprising three or more of the steps described above. In still other embodiments, the invention provides a method for preparing ent-progesterone comprising four or more of the steps described above. In certain embodiments, the Invention provides a method for preparing ent-progesterone comprising five of the steps described above.

In certain embodiments, the invention provides a method for preparing ent-progesterone in fewer than 17 linear steps. In certain embodiments, the invention provides a method for preparing ent-progesterone in fewer than 15 linear steps. In certain embodiments, the invention provides a method for preparing ent-progesterone in fewer than 13 linear steps. In certain embodiments, the invention provides a method for preparing ent-progesterone in fewer than 12 linear steps.

In another aspect, the invention provides for one or more intermediates of the synthetic method of the invention. In certain aspects, the intermediate is a compound of the formula:

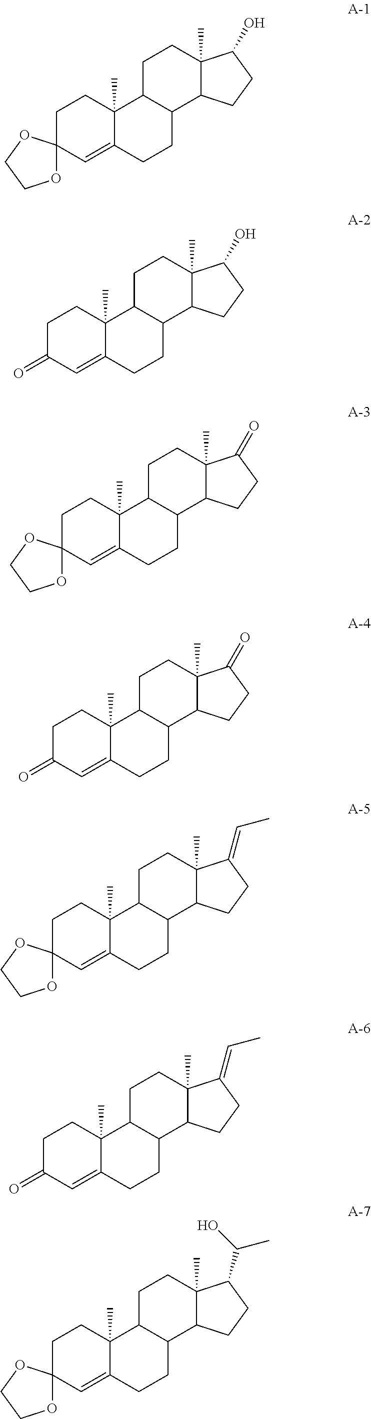

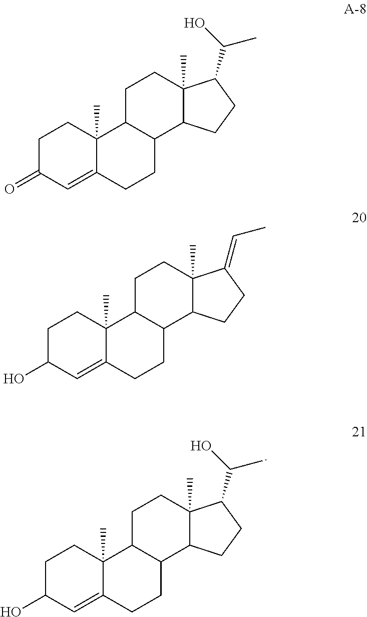 [SMILES: CC(O)[C@@H]1CCC2C3CCC4=CC(=O)CC[C@@]4(C)C3CC[C@]21C ; C/C=C1\CCC2C3CCC4=CC(O)CC[C@@]4(C)C3CC[C@@]12C ; CC(O)[C@@H]1CCC2C3CCC4=CC(O)CC[C@@]4(C)C3CC[C@]21C]

In each of the intermediates shown above, the double bond may migrate around the ring system, particularly into the second ring. For Example, intermediate A-3 may be represented as

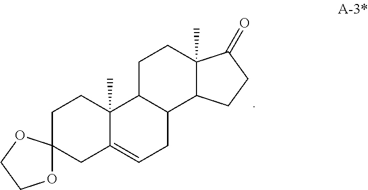 [SMILES: C[C@@]12CCC3C(CC=C4CC5(CC[C@]43C)OCCO5)C1CCC2=O]

It should be further understood that the above summary of the present invention is not intended to describe each disclosed embodiment or every implementation of the present invention. The description further exemplifies illustrative embodiments. In several places throughout the specification, guidance is provided through examples, which examples can be used in various combinations. In each instance, the examples serve only as representative groups and should not be interpreted as exclusive examples.

### DETAILED DESCRIPTION

By way of illustrating and providing a more complete appreciation of the present invention and many of the attendant advantages thereof, the following detailed description and examples are given concerning the novel synthetic synthesis for making ent-progesterone, individual novel steps within the synthetic synthesis and individual novel intermediates formed during the novel synthetic synthesis of the present invention.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, “at least one” is intended to mean “one or more” of the listed elements.

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, such as illustratively, methyl, ethyl, n-propyl 1-methylethyl(isopropyl), n-butyl, n-pentyl, and 1,1-dimethylethyl (tert-butyl).

The term “cycloalkyl” denotes a non-aromatic mono or multicyclic ring system of 3 to 12 carbon atoms such as cyclopropyl, cyclobutyl, cyclopentyl, cyclohexyl and examples of multicyclic cycloalkyl groups include perhydronapththyl, adamantyl and norbomyl groups bridged cyclic group or spirobicyclic groups e.g spiro(4,4)non-2-yl.

The term “leaving group,” or “LG”, as used herein, refers to any group that leaves in the course of a chemical reaction involving the group and includes but is not limited to halogen, brosylate, mesylate, tosylate, triflate, p-nitrobenzoate, phosphonate groups, for example.

Singular word forms are intended to include plural word forms and are likewise used herein interchangeably where appropriate and fall within each meaning, unless expressly stated otherwise.

Except where noted otherwise, capitalized and non-capitalized forms of all terms fall within each meaning.

Unless otherwise indicated, it is to be understood that all numbers expressing quantities, ratios, and numerical properties of ingredients, reaction conditions, and so forth used in the specification and claims are contemplated to be able to be modified in all instances by the term “about”.

All parts, percentages, ratios, etc. herein are by weight unless indicated otherwise.

### General Preparative Methods

The particular process to be utilized in the preparation of the compounds used in this embodiment of the present invention depends upon the specific compound desired. Such factors as the selection of the specific substituents play a role in the path to be followed in the preparation of the specific compounds of this invention. Those factors are readily recognized by one of ordinary skill in the art.

The compounds of the present invention may be prepared by use of known chemical reactions and procedures. Nevertheless, the following general preparative methods are presented to aid the reader in synthesizing the compounds of the present invention, with more detailed particular examples being presented below in the experimental section describing exemplary working examples.

The compounds of the present invention can be made according to conventional chemical methods, and/or as disclosed below, from starting materials which are either commercially available or producible according to routine, conventional chemical methods. General methods for the preparation of the compounds are given below, and the preparation of representative compounds is specifically illustrated in examples.

Synthetic transformations that may be employed in the synthesis of certain compounds of this invention and in the synthesis of certain intermediates involved in the synthesis of compounds of this invention are known by or accessible to one skilled in the art. Collections of synthetic transformations may be found in compilations, such as:
J. March. Advanced Organic Chemistry, 4th ed.; John Wiley: New York (1992);
R. C. Larock. Comprehensive Organic Transformations, 2nd ed.; Wiley-VCH: New York (1999);
F. A. Carey; R. J. Sundberg. Advanced Organic Chemistry, 2nd ed.; Plenum Press: New York (1984);
T. W. Greene; P. G. M. Wuts. Protective Groups in Organic Synthesis, 3rd ed.; John Wiley: New York (1999);
L. S. Hegedus. Transition Metals in the Synthesis of Complex Organic Molecules, 2nd ed.; University Science Books: Mill Valley, Calif. (1994);
L. A. Paquette, Ed. The Encyclopedia of Reagents for Organic Synthesis; John Wiley: New York (1994);
A. R. Katritzky; O. Meth-Cohn; C. W. Rees, Eds. Comprehensive Organic Functional Group Transformations; Pergamon Press: Oxford, UK (1995);
G. Wilkinson; F. G A. Stone; E. W. Abel, Eds. Comprehensive Organometallic Chemistry; Pergamon Press: Oxford, UK (1982);
B. M. Trost; I. Fleming. Comprehensive Organic Synthesis; Pergamon Press: Oxford, UK (1991);
A. R. Katritzky; C. W. Rees Eds. Comprehensive Heterocylic Chemistry, Pergamon Press: Oxford, UK (1984);
A. R. Katritzky; C. W. Rees; E. F. V. Scriven, Eds. Comprehensive Heterocylic Chemistry II; Pergamon Press: Oxford, UK (1996)
C. Hansch; P. G. Sammes; J. B. Taylor, Eds. Comprehensive Medicinal Chemistry. Pergamon Press: Oxford, UK (1990), each of which is incorporated herein by reference in its entirety.

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley: New York; Annual Reports in Organic Synthesis; Academic Press: San Diego Calif.; and Methoden der Organischen Chemie (Houben-Weyl); Thieme: Stuttgart, Germany. Furthermore, databases of synthetic transformations include Chemical Abstracts, each of which is incorporated herein by reference in its entirety and which may be searched using either CAS OnLine or SciFinder, Handbuch der Organischen Chemie (Beilstein), and which may be searched using SpotFire, and REACCS.

The inventive methods of the present invention to make ent-progesterone are illustrated in Reaction Schemes 1-15. The inventive methods include a number of intermediates and reaction methods which enable more efficient and less costly synthesis than heretofore known. In certain instances, reagents and solvents are listed. These reagents and solvents are exemplary and are not meant to be limited to the specific reagents or solvents shown.

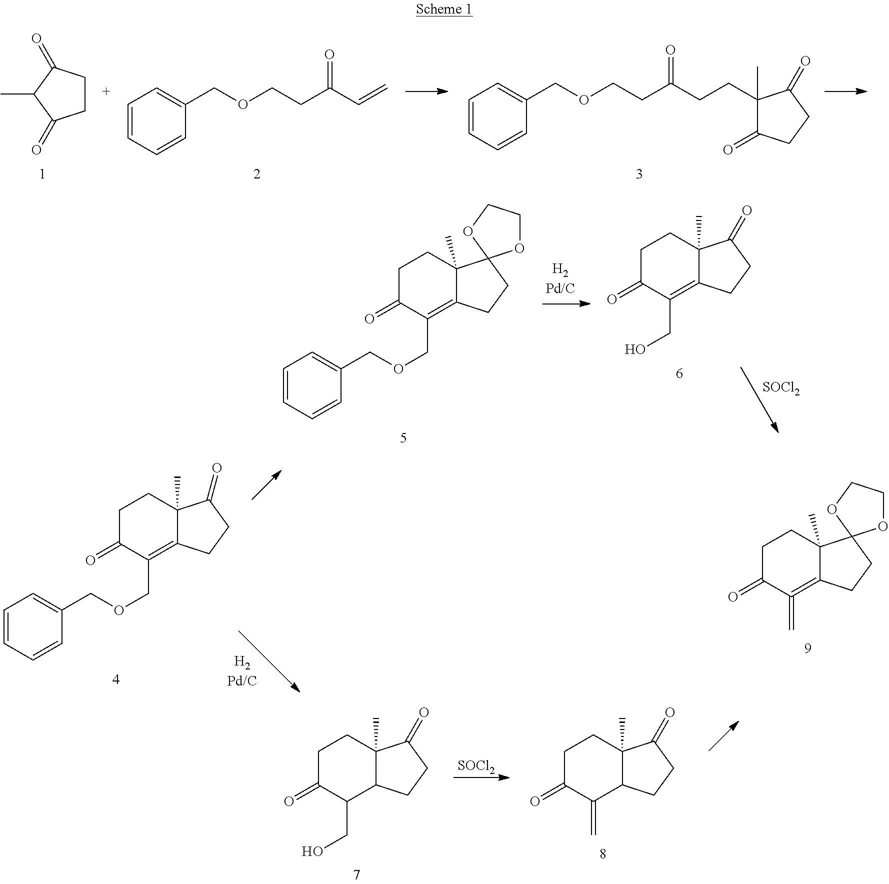

Scheme 1 represents the formation of compound (9) via two alternative processes. In Scheme 1, (1) is reacted with (2) to produce (3). The preparation of compound (2) is described in Yamauchi, Noriaki; Natsubori, Yoshiaki; Murae, Tatsushi Bulletin of the Chemical Society of Japan (2000), 73(11), 2513-2519). (3) is subjected to a stereoselective ring closing to form (4). Then (4) can be converted to (9) either: by selective protection of the carbonyl group to form (5) (as described in Bosch, M. P.; Camps, F.; Coll, J.; Guerrero, T.; Tatsuoka, T.; Meinwald, J. J. Org. Chem. 1986, 51, 773) followed by simultaneous hydrogenation of the ring double bond and cleavage of the benzyl ether to form (6) and elimination of the hydroxyl group therein with thionyl chloride; or by simultaneous hydrogenation of the ring double bond and cleavage of the benzyl ether to form (7) followed by elimination of the hydroxyl group therein with thionyl chloride to form (8) and protection of the carbonyl group (as described in Bosch, M. P.; Camps, F.; Coll, J.; Guerrero, T.; Tatsuoka, T.; Meinwald, J. J. Org. Chem. 1986, 51, 773).

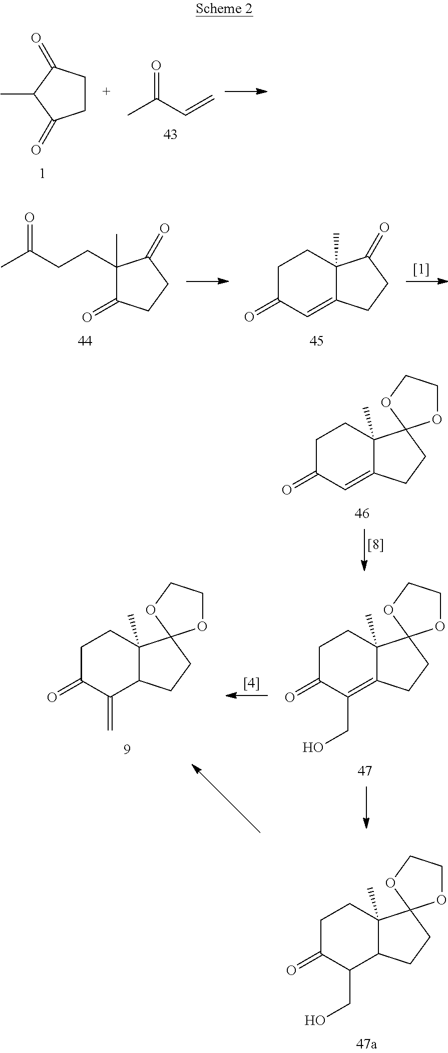

Scheme 2 represents an alternative to the formation of compound (9) of Scheme 1 from the combination of (1) and but-3-en-2-one (43). (1) and (43) are reacted to form (44) which is subjected to a stereoselective ring closing reaction to form (45). (45) is then selectively protected to form (46) (Bosch, M. P.; Camps, F.; Coll, J.; Guerrero, T.; Tatsuoka, T.; Meinwald, J. J. Org. Chem. 1986, 51, 773) which is subjected to a Baylis-Hillman reaction to form (47) (Satyanarayana reaction (Basavaiah, D.; Rao, A. J.; Satyanarayana, T. Chem. Rev. 2003, 103, 811). (47) is subjected to a Lewis acid facilitated reduction resulting in compound (9) of Scheme 1. Alternatively, (47) is hydrogenated giving (47a). Subsequent activation of the alcohol and elimination results in compound (9) of Scheme 1.

In certain embodiments, the conversion of (47a) to (9), and similar reactions, may utilize Al₂O₃ as a reagent.

One of ordinary skill in the art will recognize that activation of a beta-hydroxyketone and subsequent elimination reactions such as those described in Scheme 2 may be be accomplished under a variety of conditions including, but not limited to KOH, methanesulfonyl chloride with diisopropylethylamine, para-toluenesuffonyl chloride with dimethylaminopyridine, DCC, pyridinium hydrochloride, alumina.

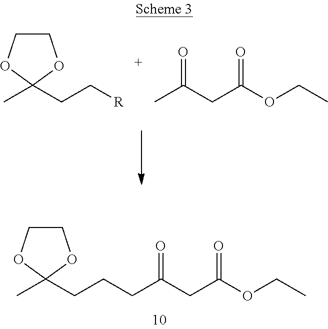

Scheme 3 represents a one step process to form compound (10) by reaction of substituted 2-ethyl-2-methyl-1,3-dioxolane a with ethyl 3-oxobutanoate. In certain embodiments, and without being limited thereto, leaving group R is —OTs, —OMs, —OTf, —Cl, —Br, or —I. In still other embodiments, leaving group R is —OTs, —Br, or —I. In yet other embodiments, leaving group R is —Br.

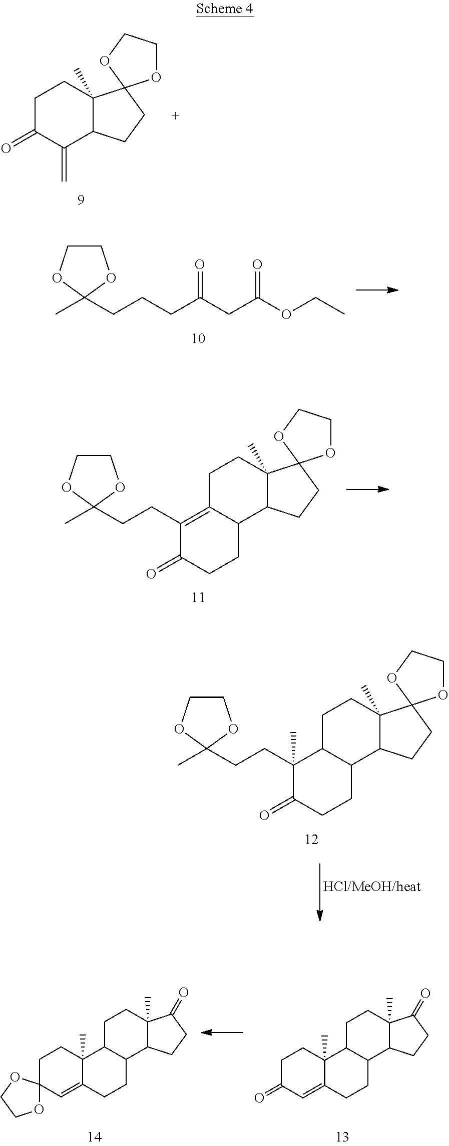

Scheme 4 represents the formation of compound (14) from the combination of (9) and (10). In Scheme 4, (9) and (10) are reacted to form (11) which is subjected to a Birch-type reduction and methylation to form (12). (12) is then double deprotected and cyclized to form (13) which is selectively reprotected to form (14) (Tsunoda, T.; Suzuki, M.; Noyorl, R. Tetrahedron Lett. 1980, 21, 1357).

In certain embodiments, the Birch-type reduction and methylation are replaced by a reductive silylation reaction followed by de-silylation and methylation.

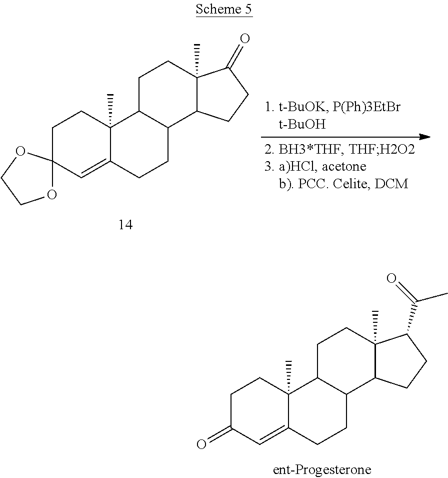 [SMILES: C[C@@]12CCC3C(CCC4=CC5(CC[C@]43C)OCCO5)C1CCC2=O ; CC(=O)[C@@H]1CCC2C3CCC4=CC(=O)CC[C@@]4(C)C3CC[C@]21C]

Scheme 5 represents the formation of ent-Progesterone from compound (14) of Scheme 4. In Scheme 5, (14) is reacted with potassium tert-butoxide and ethyl triphenylphosphonium bromide followed by hydroboration and oxidation to form ent-Progesterone. One of ordinary skill in the art will recognize that hydrolysis of the ketal protecting group can be done either before oxidation or after oxidation. One of ordinary skill in the art will further recognize that there are many reaction conditions and reagents suitable for the oxidation of an alcohol to a ketone and that alternatives to PCC include, but are not limited to, Swem, KMnO₄, Dess-Martin, TEMPO and IBX.

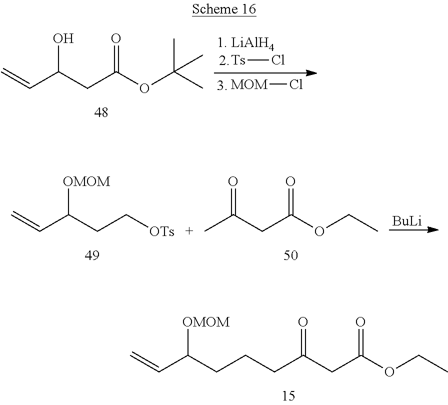

Scheme 6 represents the formation of compound (15) from the tert-butyl 3-hydroxypent-4-enoate (48) via reduction (Batt, Frederic and Fache, Fabienne, European Journal of Organic Chemistry, 2011(30), 6039-6055, S6039/1-S6039/46; 2011), formation of a tosylate and protection with a MOM (Methoxymethyl ether) protecting group to form (49). (49) is then reacted with ethyl 3-oxobutanoate (50) in the presence of a base to form (15).

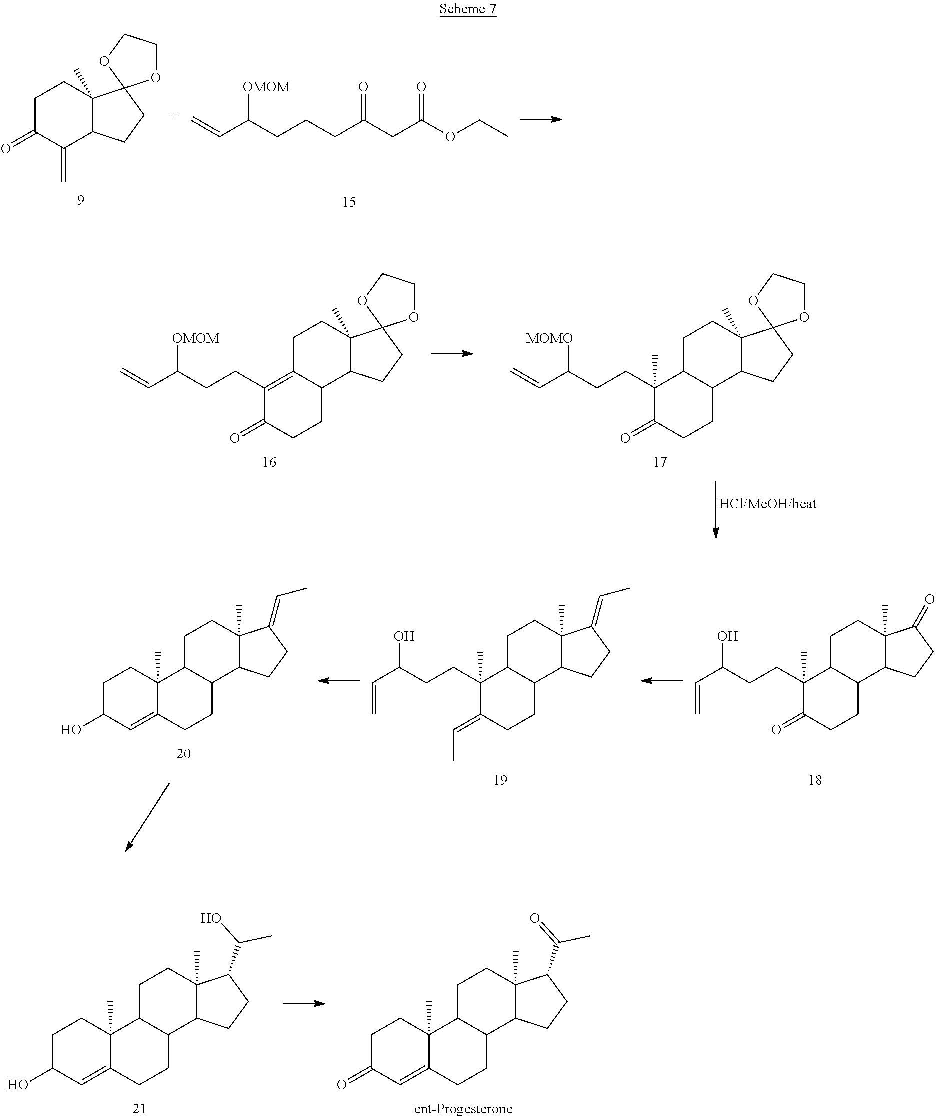

Scheme 7 represents the formation of ent-Progesterone from the combination of (9) from Scheme 1 and (15) from Scheme 6. In Scheme 7, (9) and (15) are reacted in a Robinson annulation to form (16) which is subjected to a Birch-type reduction and methylation reaction to form (17). The MOM ether and ketal of (17) are simultaneously removed to form (18) which is then subjected to a double Wittig reaction to form (19). (19) then undergoes a ring closing metasthesis reaction to form (20) which is subjected to hydroboration reaction to form (21). Double oxidation of (21) results in formation of ent-Progesterone.

In certain embodiments, the Birch-type reduction and methylation are replaced by a reductive silylation reaction followed by de-silylation and methylation.

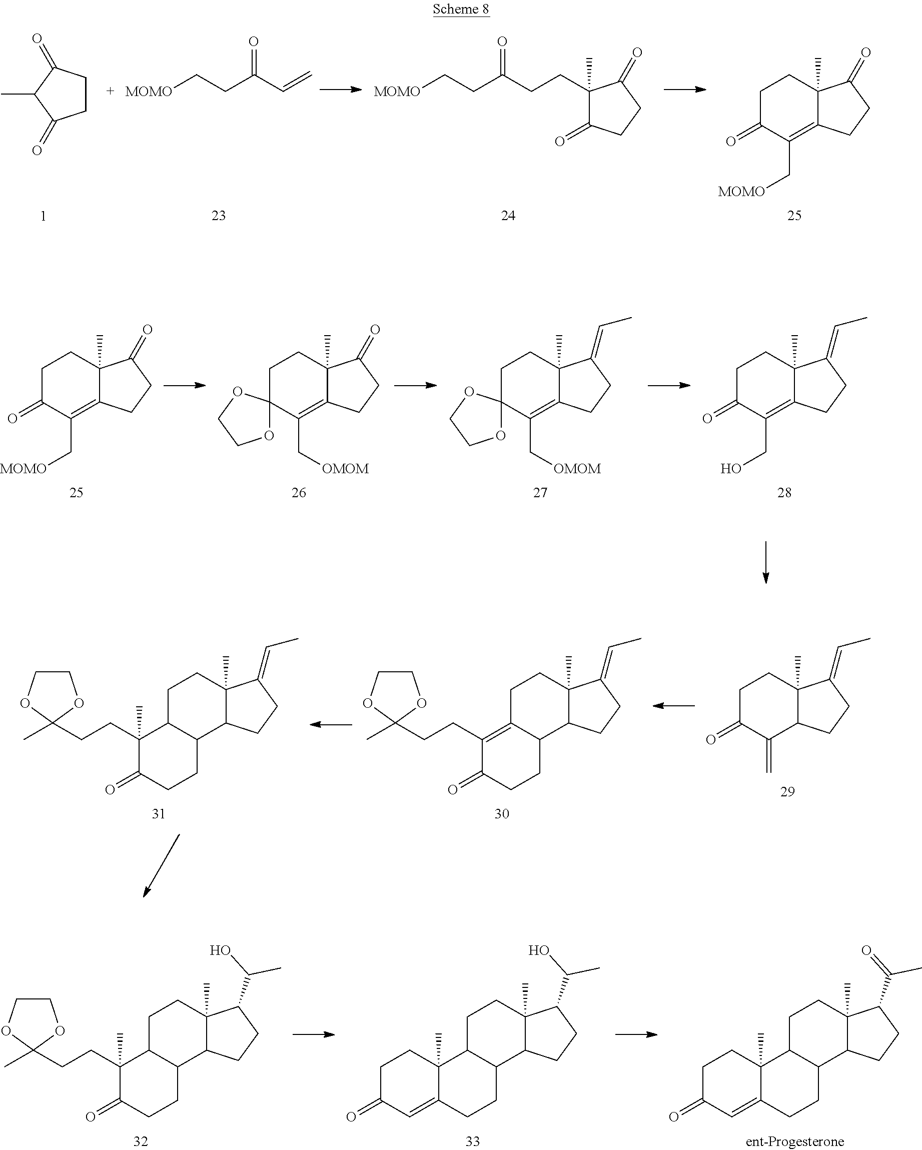

Scheme 8 represents the formation of ent-Progesterone from the combination of (1) from Scheme 1 with a methoxymethylether protected compound (23). (1) and (23) are reacted to form (24) which is subjected to a stereoselective cyclization reaction to form (25). (25) is then selectively protected to form (26) (Tsunoda, T.; Suzuki, M.; Noyori, R. Tetrahedron Lett. 1980, 21, 1357) which is subjected to a Wittig reaction with ethyl triphenylphosphonium bromide to form (27). The MOM ether and the ketal of (27) are simultaneously hydrolyzed to form (28) which is then subjected to a Lewis acid facilitated reduction to form the exocyclic double bond in (29) (Das, Biswanath; Banerjee, Joydeep; Chowdhury, Nikhil; Majhi, Anjoy; Holla, Harish, Synlett (2006), (12), 1879-1882). (29) is subjected to a Robinson annulation with (10) from Scheme 3 to form (30) which is subjected to a Birch-type reduction and methylation to form (31). (31) undergoes a hydroboration reaction to form (32). Hydrolysis of the ketal of (32) with tandem aldol cyclization forms (33). Oxidation of (33) results in ent-Progesterone.

In certain embodiments, the Birch-type reduction and methylation are replaced by a reductive silylation reaction followed by de-silylation and methylation.

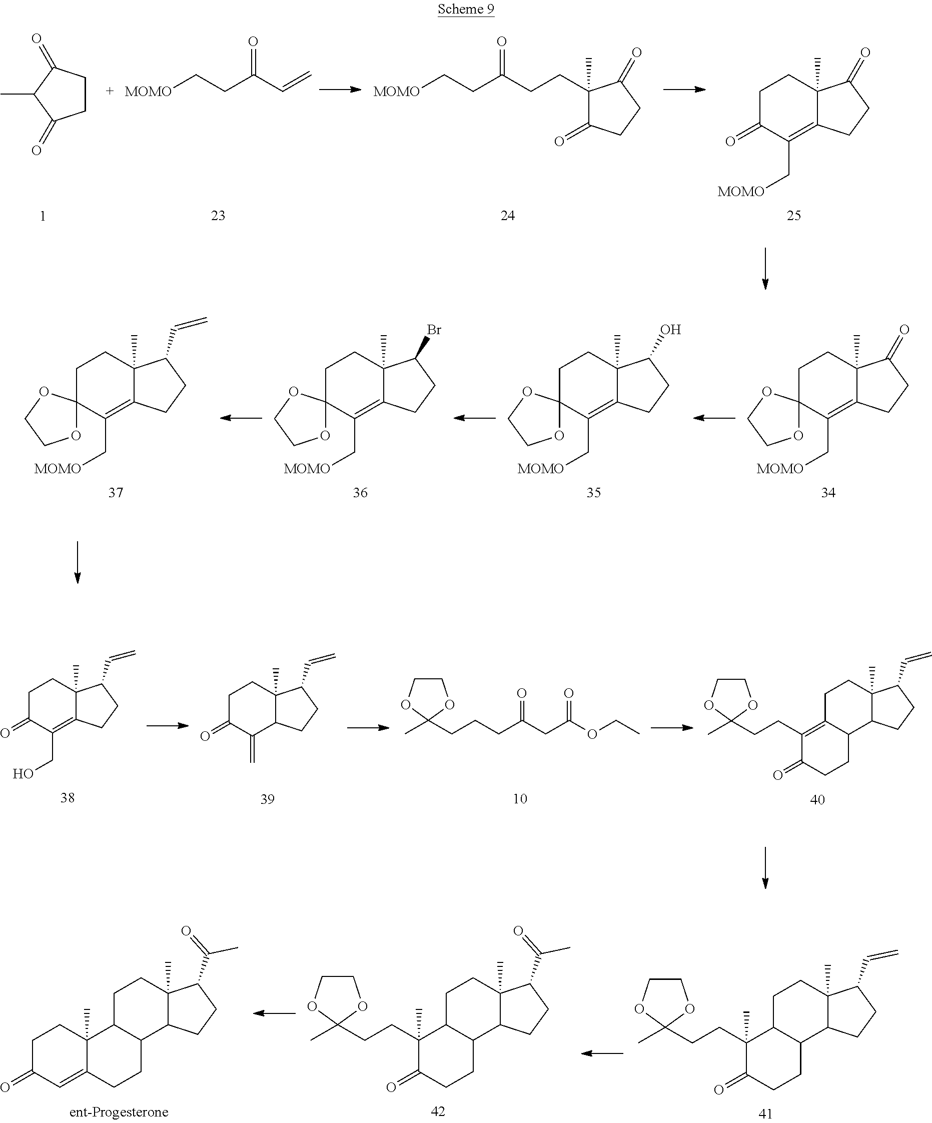

Scheme 9 represents an alternative to formation of ent-Progesterone from Scheme 8. As illustrated, compound (25) is prepared as described in Scheme 8. Continuing, compound (25) is selectively protected to produce the acetal compound (34) (Tsunoda, T.; Suzuki, M.; Noyorl, R. Tetrahedron Lett. 1980, 21, 1357) which is stereoselectively reduced to form the hydroxyl compound (35). (35) is brominated with inversion of stereochemistry to form (36) which is subjected to a nucleophilic displacement with a vinyl anion and inversion of stereochemistry to form (37). The MOM ether and ketal of (37) are simultaneously hydrolyzed to form (38) which is then subjected to Lewis acid facilitated reduction to form the exocyclic double bond in (39) (Das, Biswanath; Banerjee, Joydeep; Chowdhury, Nikhil; Majhi, Anjoy; Holla, Harish, Synlett (2006), (12), 1879-1882). (39) is reacted with compound (10) formed in Scheme 3 via a a Robinson annulation to form (40) which is subjected to a Birch-type reduction and methylation to form (41). (41) undergoes a Whacker oxidation to form (42). Tandem ketal hydrolysis and aldol cyclization of (42) results in ent-Progesterone.

In certain embodiments, the Birch-type reduction and methylation are replaced by a reductive silylation reaction followed by de-silylation and methylation.

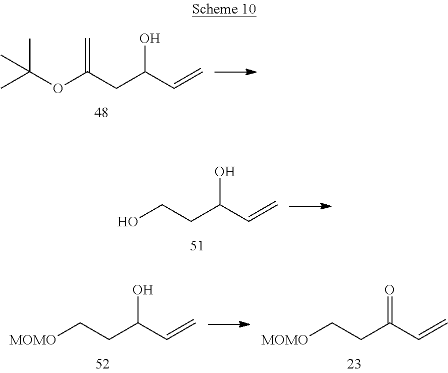

Scheme 10 represents the preparation of compound (23) illustrated in Scheme 9. This chemistry is adapted from a protocol for the preparation of a related compound (Batt, F.; Fache, F. Eur. J. Org. Chem. 2011, 6039). As illustrated, compound (48) is reduced to compound (50) (Scheme 6). The primary hydroxyl group of compound (51) (Batt, F.; Fache, F. Eur. J. Org. Chem. 2011, 6039) is then selectively converted to the corresponding methoxymethyl ether (52). Compound (52) is then oxidized to form compound (23).

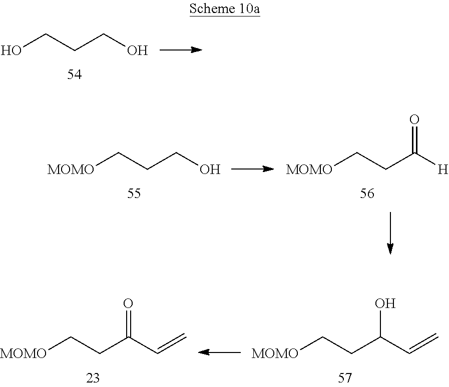

Scheme 10a represents an alternative to the preparation of compound (23) illustrated in Scheme 10. This chemistry is adapted from a protocol for the preparation of a related compound (Batt, F.; Fache, F. Eur. J. Org. Chem. 2011, 6039). As illustrated, propylene glycol is converted to its mono-methoxymethyl ether compound (55). The free hydroxyl group is then oxidized to form the aldehyde of compound (56). The aldehyde is then converted to the allylic alcohol compound (57). Compound (57) is then oxidized to form compound (23).

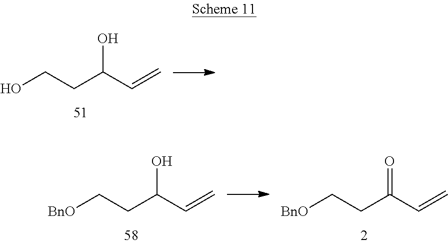

Scheme 11 represents the preparation of compound (2) illustrated in Scheme 1. This chemistry is adapted from a protocol for the preparation of a related compound (Batt, F.; Fache, F. Eur. J. Org. Chem. 2011, 6039) and represents an alternative to the synthesis described in Yamauchi, Noriaki; Natsubori, Yoshiaki; Murae, Tatsushi Bulletin of the Chemical Society of Japan (2000), 73(11), 2513-2519). As illustrated, the primary hydroxyl group of compound (51) (Batt, F.; Fache, F. Eur. J. Org. Chem. 2011, 6039) is selectively converted to the corresponding benzyl ether (58). Compound (58) is then oxidized to form compound (2).

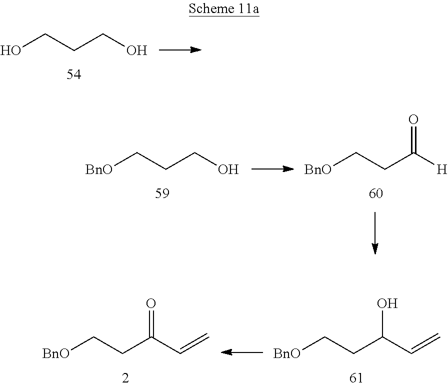

Scheme 11a represents an alternative to the preparation of compound (2) illustrated in Scheme 11. This chemistry is adapted from a protocol for the preparation of a related compound (Batt, F.; Fache, F. Eur. J. Org. Chem. 2011, 6039) and represents an alternative to the synthesis described in Yamauchi, Noriaki; Natsubori, Yoshiaki; Murae, Tatsushi Bulletin of the Chemical Society of Japan (2000), 73(11), 2513-2519). As illustrated, propylene glycol is converted to its mono-benzyl ether compound (59). The free hydroxyl group is then oxidized to form the aldehyde of compound (60). The aldehyde is then converted to the allylic alcohol compound (61). Compound (61) is then oxidized to form compound (2).

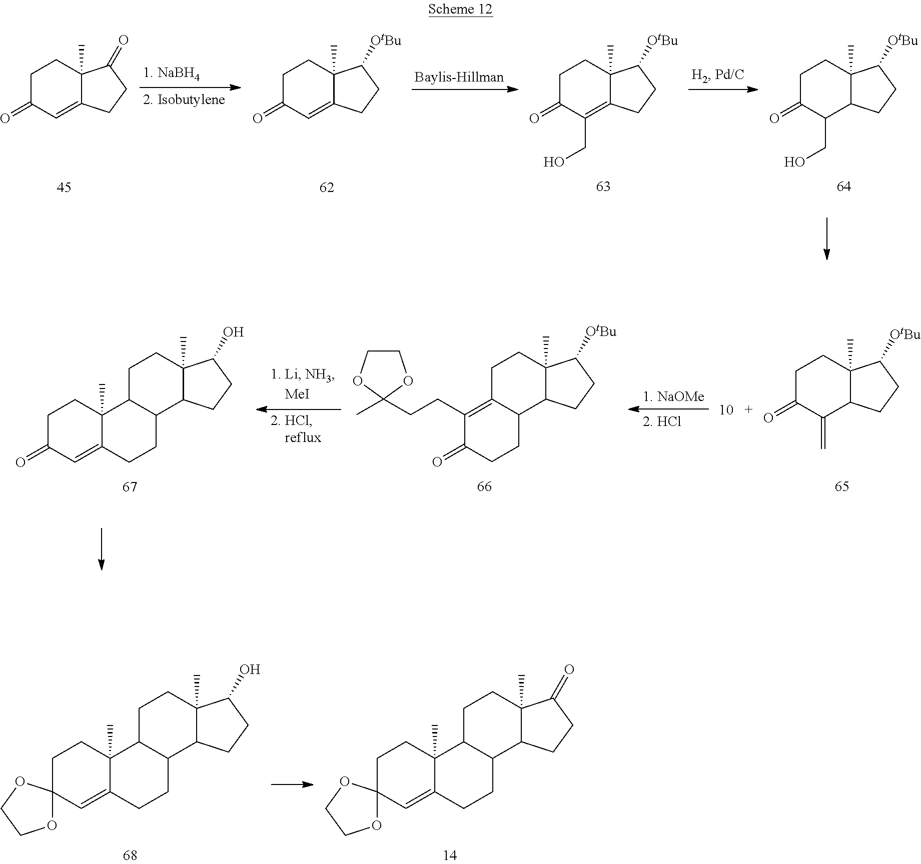

Scheme 12 provides an alternative synthesis of Compound (14) as described in Scheme 4. The synthensis includes the sequence converting compound (62) to compound (65) and the conversion of ent-testosterone (compound 67) to the dioxolane ketal compound (68).

Specifically, (45) is reduced and protected to form (62). (62) is subject to a Baylis-Hillman reaction to form (63) which is further reduced to form (64). (64) is subject to an elimination reaction to form the double bond in (65). (65) is reacted with Compound (10) from Scheme 3 to form (66) which is subjected to a Birch-type reduction and methylation followed by and cyclization to form ent-testosterone (67). ent-testosterone (67) is then ketal protected and reduced t to form (14).

In certain embodiments, the Birch-type reduction and methylation are replaced by a reductive silylation reaction followed by de-silylation and methylation.

One of ordinary skill in the art will recognize that activation of a beta-hydroxyketone and subsequent elimination reactions such as those described in Scheme 12 may be be accomplished under a variety of conditions including, but not limited to KOH, methanesulfonyl chloride with diisopropylethylamine, para-toluenesulfonyl chloride with dimethylaminopyridine, DCC, pyridinium hydrochloride, alumina.

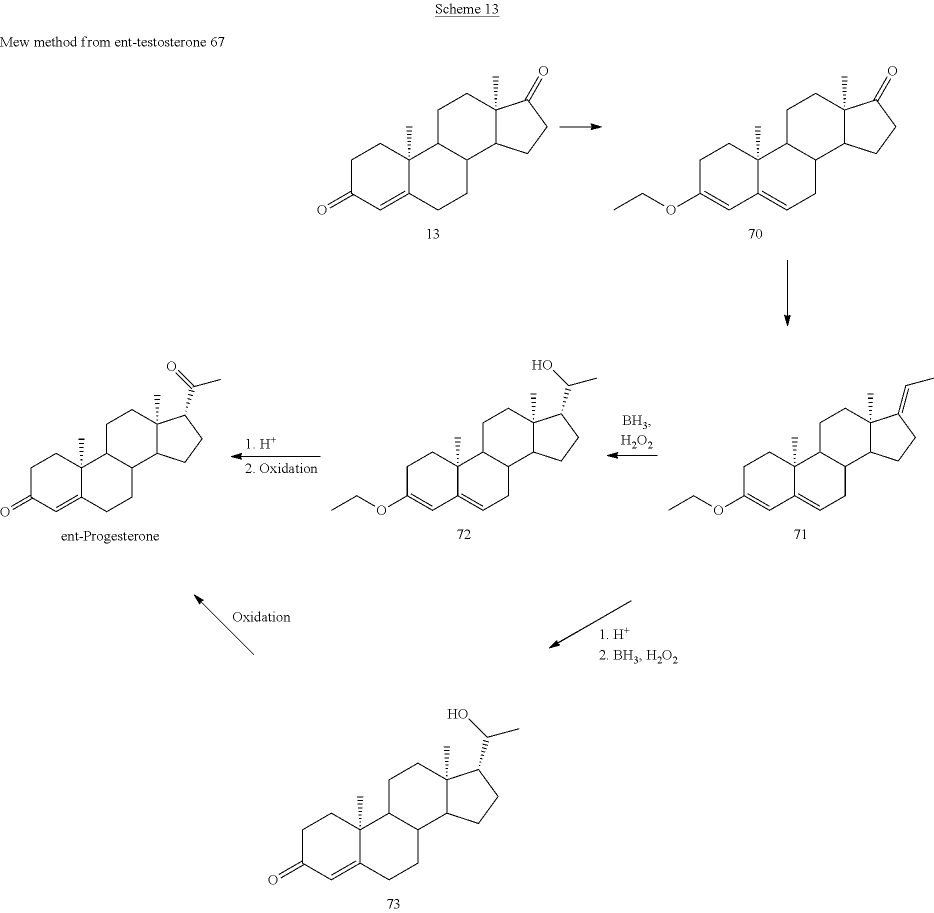 [SMILES: C[C@@]12CCC3C(CCC4=CC(=O)CC[C@]43C)C1CCC2=O ; CCOC1=CC2=CCC3C4CCC(=O)[C@]4(C)CCC3[C@]2(C)CC1 ; CC(=O)[C@@H]1CCC2C3CCC4=CC(=O)CC[C@@]4(C)C3CC[C@]21C ; CCOC1=CC2=CCC3C(CC[C@]4(C)C3CC[C@H]4C(C)O)[C@]2(C)CC1 ; C/C=C1\CCC2C3CC=C4C=C(OCC)CC[C@@]4(C)C3CC[C@@]12C ; CC(O)[C@@H]1CCC2C3CCC4=CC(=O)CC[C@@]4(C)C3CC[C@]21C]

Scheme 13 represents an alternative continuation from compound (13) (Scheme 4) and depends upon the conversion of (13) to the ethyl enol ether compound (70) followed by the Wittig reaction generating compound (71). Reactions of this type are generally described by Antimo, et al., [Steroids 77 (2012) 250-254]. This sequence can be completed by initial borane oxidation of (71) followed by hydrolysis of the enol ether and oxidation to form (72). Alternatively, (71) ether can be initially hydrolyzed followed by borane oxidation giving compound (73).

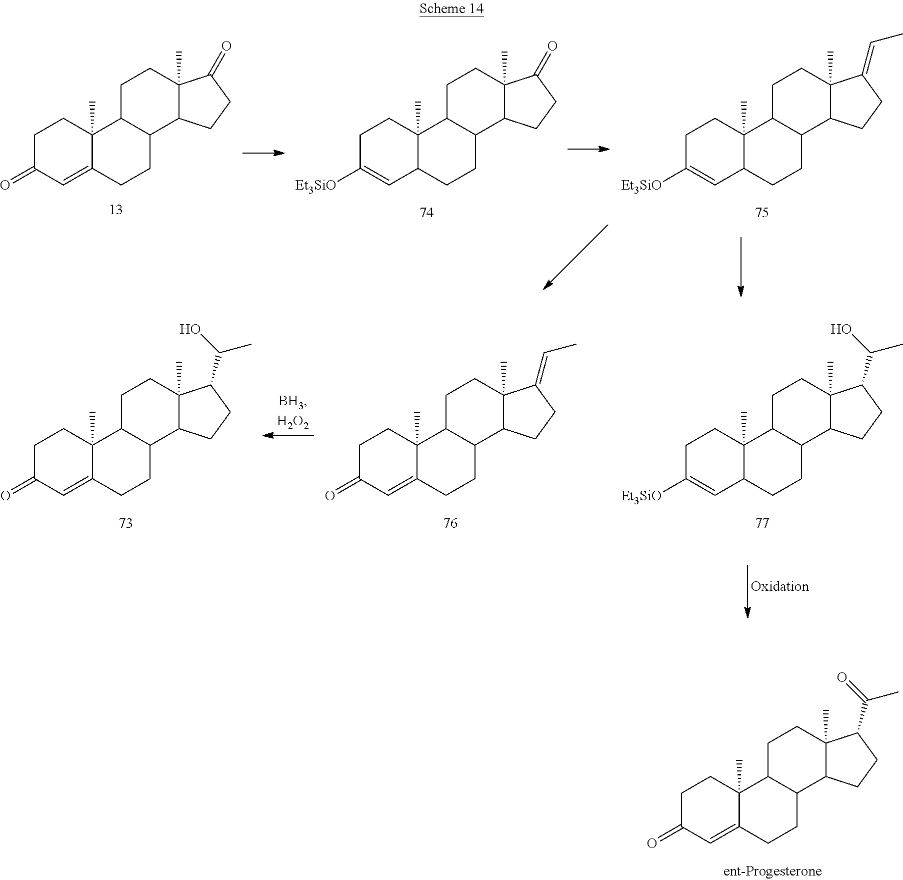

Scheme 14 represents an alternative to Scheme 13 and utilizes a reductive silylation to protect the enone of (13) to form (74). Protection of this type is generally described in Iwao, et al. [Tetrahedron Letters 49 (1972) 5085-5038] and Horiguchi, et al. [Journal of the American Chemical Society 111(16) (1989) 6259-6265]. Following borane oxidation of (75) to (77), oxidation of the alcohol and oxidative deprotection of the enone will generate ent-Progesterone. Deprotection of this type is generally described by Yoshihiko, et al. [Journal of Organic Chemistry 43(5) (1978) 1011-1013].

Alternatively, the silyl enol ether (75) can be initially oxidatively converted to (76) followed by borane oxidation to compound (73).

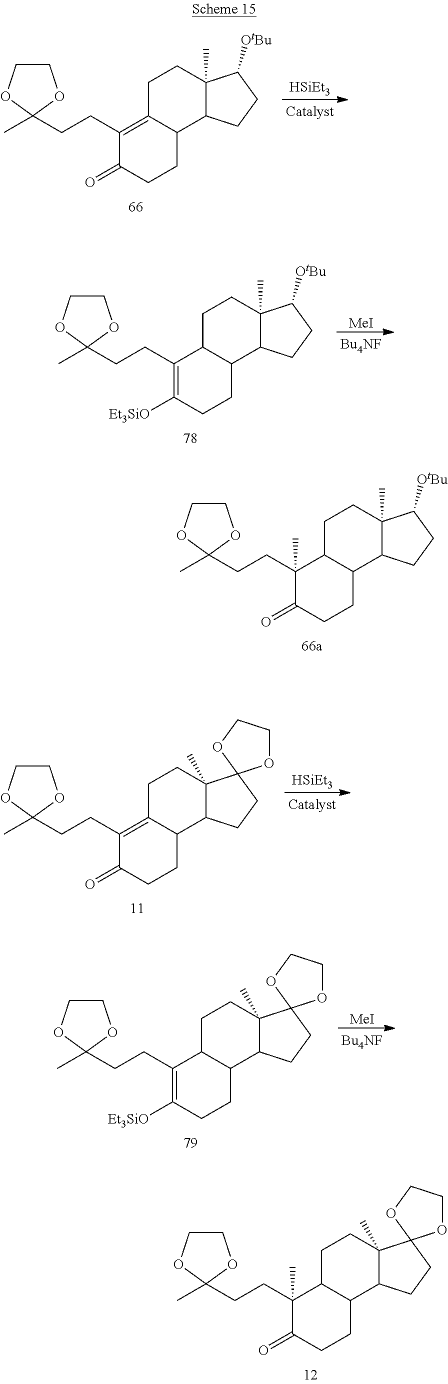

As illustrated in Scheme 4, Scheme 7, Scheme 8, Scheme 9 and in Scheme 12, all routes for the preparation of ent-progesterone involve incorporation of a methyl group as part of a Birch-type reduction alkylation sequence. This is specified in each scheme by compounds (12), (17), (30), (41) and (67), respectively. While Birch reductions generally utilize lithium dissolved in liquid ammonia, one of ordinary skill in the art will recognize that metals other than lithium may be used. Such metals include, but are not limited to, lithium, sodiium and potassium. Additionally, one of ordinary skill in the art will recognize that there are alternatives to ammonia in Birch-type reductions. Such alternatives include, but are not limited to, naphthalene and 4,4′-di-tert-butyl biphenyl. In addition to Birch-type reductions, directed reduction of an enone followed by alkylation is a useful approach for introduction of the required methyl group.

Scheme 15 illustrates this alternative as applied to (12) and compound (67). Scheme 15 may be applied to all enone compounds illustrated in each of the schemes described herein. As illustrated in Scheme 15, (66) and compound (11) are treated with triethylsilane and a catalyst to form silyl enol ethers (78) and (79), respectively. (78) and (79) are converted to compounds (66a) and (12), respectively, on treatment with tetrabutylammonium fluoride and methyl iodide. One of ordinary skill in the art will recognize that alternative silanes may be used in the reductive formation of silyl enol ethers from enones. Useful silanes include, but are not limited to, trimethylsilane, triethylsilane, trilsopropylsilane and tripropylsilane. One of ordinary skill in the art will recognize that alternative catalysts may be used in the reductive formation of silyl enol ethers from enones and trialkylsilanes. Such catalysts include, but are not limited to, Wilkinson's catalyst and other rhodium-based catalysts. One of ordinary skill in the art will recognize that multiple fluoride sources may be used for de-silylation of silyl enol ethers. Such fluoride sources include, but are not limited to, tetrabutylammonium fluoride, sodium fluoride and HF-pyridine.

The chemistry described in Scheme 15 is generally supported by Anada, et al., Kuwajima, et al., and Noyori, et al.

### Active Intermediates

The particular process described in the methods of the invention can be utilized to prepare a number of useful intermediates. In certain embodiments, the intermediates have activity separate and apart from their usefulness in the preparation of ent-Progesterone. Specifically, in certain embodiments, the active intermediate compounds have activity in the treatment of traumatic brain injury. The present invention, in certain aspects, provides a method for the treatment of traumatic brain injury comprising administering a therapeutically effective amount of an active intermediate compound to a patient in need thereof.

These active intermediate compounds include, but are not limited to,

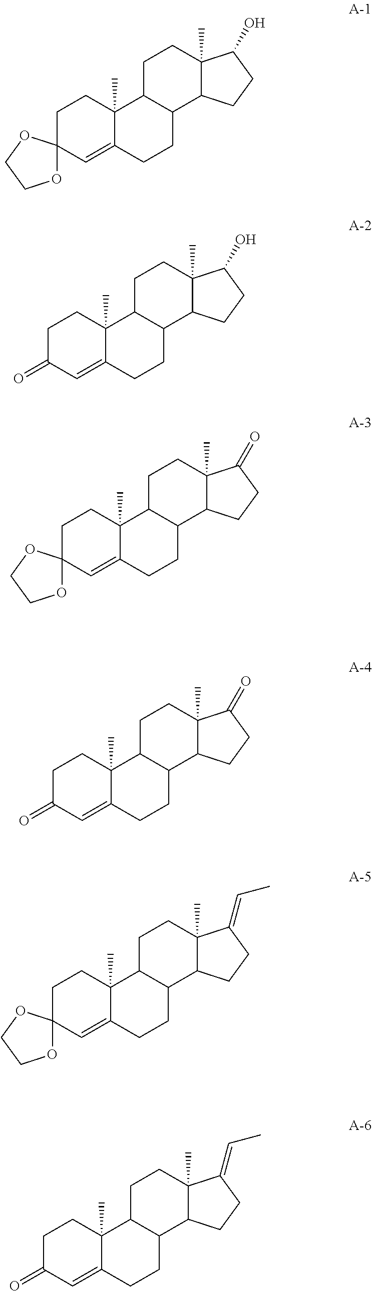 [SMILES: C[C@@]12CCC3(C=C1CCC1C2CC[C@]2(C)C1CC[C@H]2O)OCCO3 ; C[C@@]12CCC(=O)C=C1CCC1C2CC[C@]2(C)C1CC[C@H]2O ; C[C@@]12CCC3C(CCC4=CC5(CC[C@]43C)OCCO5)C1CCC2=O ; C[C@@]12CCC3C(CCC4=CC(=O)CC[C@]43C)C1CCC2=O ; C/C=C1\CCC2C3CCC4=CC5(CC[C@@]4(C)C3CC[C@@]12C)OCCO5 ; C/C=C1\CCC2C3CCC4=CC(=O)CC[C@@]4(C)C3CC[C@@]12C]

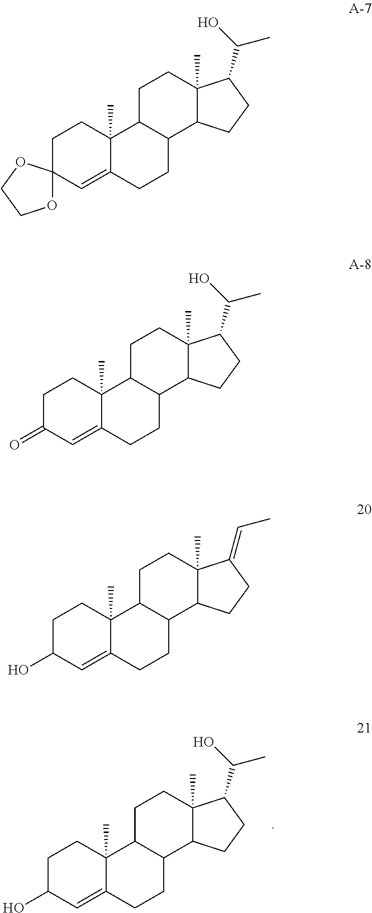 [SMILES: CC(O)[C@@H]1CCC2C3CCC4=CC5(CC[C@@]4(C)C3CC[C@]21C)OCCO5 ; CC(O)[C@@H]1CCC2C3CCC4=CC(=O)CC[C@@]4(C)C3CC[C@]21C ; C/C=C1\CCC2C3CCC4=CC(O)CC[C@@]4(C)C3CC[C@@]12C ; CC(O)[C@@H]1CCC2C3CCC4=CC(O)CC[C@@]4(C)C3CC[C@]21C]

In each of the intermediates shown above, the double bond may migrate around the ring system, particularly into the second ring. For Example, intermediate A-3 may be represented as

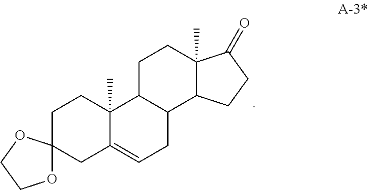 [SMILES: C[C@@]12CCC3C(CC=C4CC5(CC[C@]43C)OCCO5)C1CCC2=O]

### EXAMPLES

### Abbreviations and Acronyms

A comprehensive list of the abbreviations used by organic chemists of ordinary skill in the art appears in The ACS Style Guide (third edition) or the Guidelines for Authors for the Journal of Organic Chemistry. The abbreviations contained in said lists, and all abbreviations utilized by organic chemists of ordinary skill in the art are hereby incorporated by reference. For purposes of this invention, the chemical elements are identified in accordance with the Periodic Table of the Elements, CAS version, Handbook of Chemistry and Physics, 67th Ed., 1986-87, each of which is incorporated herein by reference in its entirety.

More specifically, when the following abbreviations are used throughout this disclosure, they have the following meanings:
atm atmosphere
br s broad singlet
Buchi rotary evaporator ®BÜCHI Labortechnik AG
C Celsius
CDCl₃ deuterated trichloromethane
Celite diatomaceous earth filter agent ®Celite Corp.
d doublet
dd doublet of doublets
DIBAL-H diisobutylaluminum hydride
DCM dichloromethane
DMI dimethyl-2-imidazolidinone
g gram
h hour, hours
¹H NMR proton nuclear magnetic resonance
HPLC high performance liquid chromatography
J coupling constant (NMR spectroscopy)
L liter
LAH lithium aluminum hydride
LG leaving group
M mol L-1 (molar)
m multiplet
MHz megahertz
min minute, minutes
mL milliliter
ρM micromolar
mol mole
MS mass spectrum, mass spectrometry
m/z mass-to-charge ratio
N equivalents L-1 (normal)
NBS N-bromo succinimide
NMO N-Methylmorpholine-N-Oxide
NMR Nuclear Magentic Resonance
pH negative logarithm of hydrogen ion concentration
q quartet
RBF round bottom flask
r.t room temperature
RT retention time (HPLC)
rt room temperature
s singlet
t triplet
THF tetrahydrofuran
TLC thin layer chromatography
TsCl tosyl chloride

The percentage yields reported in the following examples are based on the starting component that was used in the lowest molar amount. Air and moisture sensitive liquids and solutions are transferred via syringe or cannula, and are introduced into reaction vessels through rubber septa. Commercial grade reagents and solvents are used without further purification. The term “concentrated under reduced pressure” refers to use of a Buchi rotary evaporator or equivalent equipment at approximately 15 mm of Hg. All temperatures are reported uncorrected in degrees Celsius (° C.). Thin layer chromatography (TLC) is performed on pre-coated glass-backed silica gel 60 A F-254 250 μm plates.

The structures of compounds of this invention are confirmed using one or more of the following procedures.

### NMR

NMR spectra are acquired for each compound when indicated in the procedures below. NMR spectra obtained were consistent with the structures shown.

Routine one-dimensional NMR spectroscopy was performed on a 300 MHz Brucker spectrometer. The samples were dissolved in deuterated solvents. Chemical shifts were recorded on the ppm scale and were referenced to the appropriate solvent signals, such as 2.49 ppm for DMSO-d6, 1.93 ppm for CD3CN, 3.30 ppm for CD3OD, 5.32 ppm for CD2Cl2 and 7.26 ppm for CDCl3 for 1H spectra.

### Materials

Equipment used in the execution of the chemistry of this invention include but is not limited to the following:
Low temperature vacuum pump—Zhengzhouchangcheng Experimental Equipment Co., Ltd (Model #DLSB-10/20)
Rotary evaporator—Shanghaizhenjie Experimental Equipment Co., Ltd (Model #RE-52CS)
Oil pump—Shanghai Vacuum pump factory (Model #2XZ-4)
Mechanical stirrer—Beijingshijiyuhua Experimental Equipment Co., Ltd (Model #DW-3-300)
Vacuum drying oven—Beijinglianhekeyi Experimental Equipment Co., Ltd (Model #DZF-6020)
LCMS—Agilent (Model #1200-6100)
GCMS—Agilent (Model #7890A-5975C)
GC—Agilent (Model #7890A)
Chiral HPLC—Shimadzu (Model #LC-20AT)
NMR—Bruker (Model #AVANCEII300)
Liquid chromatorgraph—Agilent (Model #G1322A)
High temperature oil bath—SMS (Model #CC508)
Electronic balance—LBTEC (Model #XS205DU)

Chemicals and solvents that are used in the experimental workups are purchased from either Sigma Aldrich, Fisher Scientific or EMD unless otherwise stated and the solvents used are either ACS or HPLC grade with the two grades being used interchangeably. For TLC analysis, the silica 60 gel glass backed TLC plates are used.

### Preparation of Compound 3 (Scheme 1)

2-Methyl-1,3-pentanedione (1 g, 1.2 eq.) was dissolved in anhydrous acetonitrile (40 mL) and 5-benzyloxy-pent-1-ene-2-one (1.5 g, 1.0 eq.) was added followed by triethylamine (50 mg, 0.05 eq.). The reaction was stirred at 25-30 deg C. for 12 hours after which, it was concentrated to dryness. Purification of the residue on silica gel (Ethyl acetate/Hexane 1/5) gave compound 3 (1.8 g) as a colorless oil. 1H NMR (300 MHz, CDCl3): δ 1.10 (s, 3H), 1.90 (t, 2H), 2.50 (t, 2H), 2.65 (t, 2H), 2.70-2.90 (m, 4H), 3.70 (t, 2H), 4.50 (s, 2H), 7.25-7.4 (m, 5H). MS (M++1) 303.1.

### Preparation of Compound 46 (Scheme 2)

2-Ethyl-2-methyl-1,3-dioxolane (120 mL) and compound 45 (20 g, 1.0 eq.) were combined under nitrogen. Ethylene glycol (1.2 mL, 0.14 eq.) was added followed by p-toluenesulfonic acid (390 mg, 0.02 eq.). The reaction was stirred at 25-30 deg C. for 96 hours until the concentration of compound 45 was less than 20% as measured by HPLC. Ethyl acetate (100 mL) was added and the resulting mixture was washed with water (2×100 mL), dried over anhydrous sodium sulfate, filtered and concentrated to dryness. The residue was purified on silica gel (ethyl acetate/hexane 1/20) yielding compound 46 (8 g) as a colorless oil. 1H NMR (300 MHz, CDCl3): δ 1.20-1.35 (m, 7H), 1.60-1.70 (m, 1H), 1.90-2.00 (m, 1H), 2.10-2.80 (m, 6H), 3.85-4.05 (m, 4H), 5.85 (s, 1H). MS (M++1) 209.1.

### Preparation of Compound 47 (Scheme 2)

Compound 46 (8.0 g, 1.0 eq.) was added to a mixture of 1,4-dioxane (40 ml) and water (34 mL). Formaldehyde (3.1 g, 1.0 eq.) was then added followed by 1,4-diazabicyclo[2.2.2]octane (DABCO, 8.5 g, 1.0 eq). The reaction was stirred at 25-30 deg C. for 120 hours after which, ethyl acetate (100 mL) was added. The mixture was washed with water (2×100 mL), dried over anhydrous sodium sulfate, filtered and concentrated to dryness. Purification of the residue on silica gel (10% ethyl acetate in hexane) gave compound 47 (5 g) as a colorless oil. 1H NMR (300 MHz, CDCl3): δ 1.25 (m), 1.65 (m, 1H), 1.95 (m, 1H), 2.15-2.80 (m), 3.90-4.05 (m), 5.80 (s, 1H).

### Preparation of Compound 47a (Scheme 2)

Compound 47 (2 g) was dissolved in anhydrous tetrahydrofuran (THF, 200 mL) under a nitrogen atmosphere. 10% Pd/C (200 mg) was added and the reaction was placed under a hydrogen atmosphere. The reaction was stirred at −10-0 deg C. over 40 hours after which, the Pd/C was removed by filtration. The filtrate was concentrated to dryness and the residue was purified on silica gel (10% ethyl acetate/hexane) giving compound 47a (1.6 g) as a colorless oil. 1H NMR (300 MHz, DMSO-d6): δ 0.95-1.15 (m, 1H), 1.55-2.10 (m), 2.50 (t, 2H), 2.40-2.50 (m, 1H), 2.70-2.80 (q, 1H), 3.15-3.30 (m, 1H), 3.65-3.90 (m), 4.35 (dd, 1H). MS (M++1) 241.1.

### Preparation of Compound 9 (Scheme 2)

Compound 47a (300 mg, 1.0 eq.) was dissolved in dichloromethane (DCM, 3 mL) and triethylamine (TEA, 3.0 eq.) was added. The mixture was cooled to −10 deg C. under nitrogen and methanesulfonyl chloride (1.2 eq.) was added dropwise. Stirring was continued at 10-20 deg C. for 4 hours after which, toluene (3 mL) was added followed by 1,8-diazabicycloundec-7-ene (DBU, 3.0 eq.). Stirring was continued at 25-30 deg C. for an additional 40 hours after which, the reaction was washed with water (2×3 mL), dried over anhydrous sodium sulfate, filtered and concentrated to dryness. The residue was purified on silica gel (ethyl acetate/hexane 1/10) giving compound 9 (100 mg) as a colorless oil. 1H NMR (300 MHz, DMSO-d6): δ 1.00 (s, 3H), 1.40-1.60 (m, 2H), 1.70-2.00 (m, 4H), 2.30-2.55 (m, 2H), 2.80 (m, 1H), 3.80-3.95 (m, 4H), 5.20 (s, 1H), 5.70 (s, 1H). MS (M++1) 223.1.

### Preparation of Compound 10 (Scheme 3)

Sodium hydride (426 mg, 1.2 eq.) was placed under nitrogen and cooled to 0 deg C. Tetrahydrofuran (THF, 10 mL) was added followed by hexamethylphosphoramide (HMPA, 326 mg, 0.25 eq.). Ethyl acetoacetate (1 mL, 1.0 eq.) was added and the mixture was stirred at 0 deg C. for 10 minutes. n-Butyllithium (2.5M, 3.6 mL, 1.1 eq.) was added and the mixture was stirred at 0 deg C. for an additional 10 minutes. 2-(2-methyl-1,3-dioxolan-2-yl)ethylbromide (1.6 g, 1.0 eq.) was added and the reaction was stirred at 0 deg C. for 30 minutes. The reaction was quenched with aqueous oxalic acid (10%, 20 mL) and washed with dichloromethane (DCM, 3×20 mL). The organic phase was additionally washed with saturated aqueous sodium bicarbonate (30 mL) and brine (30 mL). The organic phase was dried over anhydrous sodium sulfate, filtered and concentrated. The residue was purified on silica gel (ethyl acetate/hexane 1/30) giving compound 10 (600 mg) as a yellow oil. 1H NMR (300 MHz, DMSO-d6): δ 1.25 (t, 3H), 1.30 (s, 3H), 1.60-1.80 (m, 4H), 2.60 (t, 2H), 3.45 (s, 2H), 3.90-4.00 (m, 4H), 4.15-4.25 (q, 2H).

### Preparation of Compound 11 (Scheme 4)

Compound 9 (500 mg, 1.0 eq.) was dissolved in methanol (15 mL) and compound 10 (715 mg, 1.3 eq.) was added. Sodium methoxide (0.2 eq) was added and the mixture was stirred at 30 deg C. for 16 hours. Aqueous sodium hydroxide (5 M, 5.0 eq.) was added and the reaction was stirred for an additional 4 hours at 30 deg C. The methanol was then removed utilizing a rotary evaporator. Water (5 mL) was then added and the mixture was washed with toluene (2×3 mL). The aqueous phase was cooled to 0 deg C. and acidified to pH 6 with aqueous HCl (6 N). The mixture was washed with ethyl acetate and the organic extract was concentrated to dryness. The residue was purified on silica gel (ethyl acetate/hexane 1/10) giving compound 11 (150 mg) as a colorless oil. MS (M++1) 377.1.

### Preparation of Compound 48 (Scheme 6)

Compound 48 was prepared as described by Batt, et al. (Eur. J. Org. Chem., 2011, 6039-6055).

### Preparation of Compound 49 (Scheme 6)

Compound 48 (100 g) was reduced to the corresponding alcohol using lithium aluminum hydride as described by Batt, et al. (Eur. J. Org. Chem., 2011, 6039-6055). The resulting diol (1 g, 1.0 eq.) was dissolved in dichloromethane (DCM, 10 mL) under nitrogen. Triethylamine (2.0 eq.) was added and the resulting mixture was cooled to 0 deg C. Para-toluenesuffonyl chloride (1.0 eq.) was added slowly and the reaction was stirred at 0 deg C. for 30 minutes. The resulting mixture was washed with water (10 mL) after which, it was dried over anhydrous sodium sulfate, filtered and concentrated to dryness. The residue was purified on silica gel (ethyl acetate/hexane 1/10) giving the desired primary tosylate (500 mg) as a yellow oil. The resulting primary tosylate (100 mg, 1.0 eq.) was dissolved in DCM (10 mL) under nitrogen. Diisopropylethyl amine (DIEA, 1.2 eq.) was added and the mixture was cooled to 0 deg C. Methoxymethyl chloride (1.0 eq) was added dropwise and the reaction was stirred from 0-25 deg C. over 2 hours after which, it was washed with water (10 mL). The organic phase was dried over anhydrous sodium sulfate, filtered and concentrated to dryness. The residue was purified on silica gel (Ethyl acetate/hexane 1/20) giving the desired compound 49 (60 mg) as a yellow oil.

### Preparation of Compound 24 (Scheme 9)

2-Methyl-1,3-cyclopentanedione (3.0 g, 1.2 eq.) was combined with compound 23 (3.1 g, 1.0 eq.) and acetonitrile (ACN, 30 mL). Triethylamine (TEA, 110 mg, 0.05 eq) was added and the reaction was stirred at 25 deg C. for 4 hours. Dichloromethane (DCM, 100 mL) was then added and the mixture was washed with aqueous hydrochloric acid (2×30 mL) and saturated aqueous sodium bicarbonate (2×30 mL). The organic phase was dried over anhydrous sodium sulfate, filtered and concentrated to dryness. The residue was purified on silica gel (ethyl acetate/hexane 1/30) giving compound 24 (2.6 g) as a yellow oil. 1H NMR (300 MHz, CDCl3): δ 1.10 (s, 3H), 1.90 (t, 2H), 2.50 (t, 2H), 2.65 (t, 2H), 2.70-2.90 (m, 4H), 3.35 (s, 3H), 3.75 (t, 2H), 4.60 (s, 2H).

### Preparation of compound 52—5-Methoxymethoxy-pent-1-ene-3-ol (Scheme 10)

Compound 48 (100 g) was reduced to the corresponding alcohol using lithium aluminum hydride as described by Batt, et al. (Eur. J. Org. Chem., 2011, 6039-6055). The resulting diol (13 g, 1 eq.) was added to a mixture of cyclohexane (26 mL), dichloromethane (DCM, 13 mL) and diisopropyl ethylamine (DIEA, 18 g, 1.1 eq.) under nitrogen. Methoxymethyl chloride (1 eq.) was added dropwise and the reaction was stirred at 20 deg C. for 12 hours. DCM (100 mL) was then added and the mixture was washed with aqueous hydrochloric acid (2 M, 30 mL) and saturated aqueous sodium bicarbonate (2×30 mL). The organic phase was dried over anhydrous sodium sulfate, filtered and concentrated to dryness. The residue was purified on silica gel (10% ethyl acetate/hexane) giving the primary MOM ether (compound 52, 4 g) as a yellow oil. 1H NMR (300 MHz, CDCl3): δ 1.75-1.95 (m, 2H), 3.35 (s, 3H), 3.65-3.80 (m, 2H), 4.30-4.35 (m, 1H), 4.65 (s, 2H), 5.10-5.15 (m, 1H), 5.25-5.30 (m, 1H), 5.85-5.95 (m, 1H).

### Preparation of compound 23—5-Methoxymethoxy-pent-1-ene-3-one (Scheme 10)

Compound 52 (3.5 g, 1.0 eq.) was dissolved in dimethyl sulfoxide (DMSO, 20 mL) under nitrogen. 2-lodoxybenzoic acid (IBX, 9.8 g, 1.5 eq.) was added and the reaction was stirred at 20 deg C. for 12 hours. DCM (100 mL) was added and the resulting mixture was washed with saturated aqueous sodium sulfite (30 mL) and saturated aqueous sodium bicarbonate (30 mL). The organic phase was dried over anhydrous sodium sulfate, filtered and concentrated to dryness. The residue was purified on silica gel (Ethyl acetate/hexane 1/30) giving the desired compound 23 (3.1 g) as a yellow oil. 1H NMR (300 MHz, CDCl3): δ 2.90 (t, 2H), 3.35 (s, 3H), 3.90 (t, 2H), 4.65 (s, 2H), 5.90 (d, 1H), 6.20-6.45 (m, 2H).

### Preparation of compound 55 (Scheme 10a)—3-Methoxymethylpropan-1-ol

Cyclohexane (180 mL), dichloromethane (90 mL) and diisopropylethylamine (34 g, 1.1 eq.) were combined and propane-1,3-diol (20 g, 1.0 eq.) was added. Methoxymethyl chloride (20.9 g, 0.99 eq.) was added dropwise maintaining the internal reaction temperature at 20 deg C. The reaction was stirred at 20 deg C. for 12 hours after which, dichloromethane (100 mL) was added. The mixture was washed with saturated aqueous sodium bicarbonate (2×30 mL), dried over anhydrous sodium sulfate, filtered and concentrated to dryness. The residue was purified on silica gel (ethyl acetate/hexane 1/5) giving compound 55 (5 g) as a yellow oil. 1H NMR (300 MHz, CDCl3): δ 1.80-1.90 (m, 2H), 3.40 (s, 3H), 3.70 (t, 2H), 3.80 (t, 2H), 4.65 (s, 2H).

### Preparation of compound 56 (Scheme 10a)—3-Methoxymethylpropionaldehyde

Compound 55 (1 g, 1.0 eq.) was dissolved in dimethylsulfoxide (10 mL) and 2-lodoxybenzoic acid (IBX, 3.5 g, 1.5 eq.) was added. The reaction was stirred at 20 deg C. for 12 hours after which, it was washed with saturated aqueous sodium sulfite (20 mL) and saturated aqueous sodium bicarbonate (20 mL). The organic phase was dried over anhydrous sodium sulfate, filtered and concentrated to dryness. The residue was purified on silica gel (ethyl acetate/hexane 1/20) giving compound 56 (0.3 g, 60% purity) as a yellow oil. 1H NMR (300 MHz, CDCl3): δ 1.80-1.90 (m, 2H), 3.40 (s, 3H), 3.70 (t, 2H), 3.80 (t, 2H), 4.65 (s, 2H).

### Preparation of Compound 2 (Scheme 11)

Compound 2 is reported by Yamauchi, et al. (Bull. Chem. Soc. Jpn., 2001, 2513-2519). The Scheme 11 sequence for preparation of compound 2 was adapted from Batt, et al. (Eur. J. Org. Chem., 2011, 6039-6055).

### Preparation of Compound 2 (Scheme 11a)

Propylene glycol (500 g) was combined with benzyl bromide (100 g, 1.0 eq.) under nitrogen. Sodium hydroxide (28 g, 1.2 eq.) was added and the mixture was stirred at 20 deg C. for 4 hours. Ethyl acetate (800 mL) was then added and the mixture was washed with water (500 mL). The organic phase was dried over anhydrous sodium sulfate, filtered and concentrated to dryness giving the desired crude 3-benzyloxypropanol (100 g) as a yellow oil. 1H NMR (300 MHz, CDCl3): δ 1.85-1.90 (m, 2H), 3.65 (t, 2H), 3.80 (t, 2H), 4.25 (t, 1H), 4.55 (s, 2H), 7.25-7.40 (m, 5H). Crude 3-benzyloxypropanol (100 g, 1.0 eq.) was combined with dimethyl sulfoxide (DMSO, 500 mL) and tetrahydrofuran (THF, 500 mL) under nitrogen. 2-lodoxybenzoic acid (IBX, 253 g, 1.5 eq.) was added and the reaction was stirred at 20 deg C. for 12 hours. Ethyl acetate (1500 mL) was then added and the mixture was washed with saturated aqueous sodium sulfite (500 mL) and saturated aqueous sodium bicarbonate (500 mL). The organic phase was washed with anhydrous sodium sulfate, filtered and concentrated to dryness. The residue was purified on silica gel (ethyl acetate/hexane 1/20) giving the desired 3-benzyloxypropionaldehyde (30 g) as a yellow oil. 1H NMR (300 MHz, CDCl3): δ 2.70 (m, 2H), 3.80 (t, 2H), 4.55 (s, 2H), 7.25-7.40 (m, 5H), 9.80 (s, 1H). 3-benzyloxypropionaldehyde (30 g, 1.0 eq.) was dissolved in THF under nitrogen and cooled to 0 deg C. Vinylmagnesium bromide (1M, 220 mL, 1.2 eq.) was added and the reaction was stirred at 0 deg C. for 1 hour. Saturated aqueous ammonium chloride (100 mL) was then added and the mixture was extracted with dichloromethane (DCM, 3×100 mL). The organic extracts were dried over anhydrous sodium sulfate, filtered and concentrated to dryness giving crude 5-benzyloxy-pent-1-ene-3-ol. 1H NMR (300 MHz, CDCl3): δ 1.75-1.99 (m, 2H), 3.60-3.75 (m, 2H), 4.30-4.40 (m, 1H), 4.50 (s, 2H), 4.70 (s, 1H), 5.10-5.15 (m, 1H), 5.25-5.30 (m, 1H), 5.80-5.95 (m, 1H), 7.25-7.40 (m, 5H). This material was dissolved in DMSO (120 mL) and THF (120 mL) under nitrogen and IBX (65 g, 1.5 eq.) was added. The mixture was stirred at 20 deg C. for 12 hours after which, ethyl acetate (500 mL) was added. The resulting mixture was washed with saturated aqueous sodium sulfite (200 mL) and saturated aqueous sodium bicarbonate (200 mL). The organic phase was dried over anhydrous sodium sulfate, filtered and concentrated to dryness. The residue was purified on silica gel (ethyl acetate/hexane 1/20) giving the desired 5-benzyloxy-pent-1-ene-3-one (12.7 g) as a yellow oil. 1H NMR (300 MHz, CDCl3): δ 2.95 (t, 2H), 3.80 (t, 2H), 4.55 (s, 3H), 5.85 (d, 1H), 6.20-6.40 (m, 2H), 7.20-7.40 (m, 5H).

### REFERENCE LIST

1. US Publication No. US 2005/0187188 to Stein et al., published Aug. 25, 2005-“Methods for the Treatment of a Traumatic Central Nervous System Injury”
2. US Publication No. US 2007/0078117 to Hoffman et al., published Apr. 5, 2007-“Methods for the Treatment of a Traumatic Central Nervous System Injury”
3. US Publication No. US 2008/0318914 to Hoffman et al., published Dec. 25, 2008—“Methods for the Treatment of a Traumatic Central Nervous System Injury”
4. US Publication No. US 2009/0221544 to Stein et al., published Sep. 3, 2009—“Methods for the Treatment of a Traumatic Central Nervous System Injury via a Tapered Administration Protocol”
5. US Publication No. US 2009/0325920 to Hoffman et al., published Dec. 31, 2009—“Methods for the Treatment of a Traumatic Central Nervous System Injury”
6. US Publication No. US 2011/0306579 to Stein et al., published Dec. 15, 2011—“Methods of Neuroprotection using Neuroprotective Steroids and a Vitamin D”
7. R. J. Auchus et al. “The Enantiomer of Progesterone (ent-progesterone) is a Competitive Inhibitor of Human Cytochromees P450c17 and P450c21,” Archives of Biochemistry and Biophysics 409 (2003) 134-144
8. S. D. Rychnovsky et al. “Synthesis of ent-Cholesterol, the Unnatural Enantiomer,” J. Org. Chem. 57 (1992) 2732-2736
9. S. Talengaonkar et al. “Intranasal Delivery: An Approach to Bypass the Blood Brain Barrier,” Indian J. Pharmacol, Vol 36, Issue 3, (2004), 140-147
10. H. Nemoto et al. “First Enantioselective Total Synthesis of (+)-Cortisone,” J. Org. Chem. 55 (1990) 5625-5631
11. W. S. Johnson et al. “Synthesis of dl-Progesterone,” Journal of the American Chemical Society, Vol 93, Issue 17, (1971) 4332-4334
12. M. Weimar et al. “Enantioselective Synthesis of (+)-Estrone Exploiting a Hydrogen Bond-Promoted Diels-Alder Reaction,” J. Org. Chem 75 (2010) 2718-2721
13. Herrmann et al. “Formal Total Synthesis of (±)-Estrone and Zirconocene-Promoted Cyclization of 2-Fluoro-1-7-octadienes and Ru-Catalyzed Ring Closing Metathesis,” J. Org. Chem 73 (2008) 6202-6206
14. Q. Hu et al. “Simple, Catalytic Enantioselective Synthesis of Estrone and Desogestrel,” J. Am Chem Soc 126 (2004) 5984-5986
15. Y. Horiguchi et al. “Total Synthesis of (±)-Cortisone. Double Hydroxylation Reaction for the Construction of Corticoid Side Chain,” J. Org. Chem. 51 (1986) 4323-4325
16. U.S. patent application Ser. No. 13/645,854 to VanLandingham et al. “Prophylactic and Post-Acute Use of Progesterone to Better Outcomes Associates with Concussion”
17. U.S. patent application Ser. No. 13/645,881 to VanLandingham et al. “Nasal Delivery Mechanism for Prophylactic and Post-Acute Use for Progesterone and/or Its Enantiomer for Use in Treatment of Mild Traumatic Brain Injuries”
18. U.S. patent application Ser. No. 13/645,925 to VanLandingham et al. “Prophylactic and Post-Acute Use of Progesterone in Conjunction with its Enantiomer for Use in Treatment of Mild Traumatic Brain Injuries”
19. International PCT Patent Application No. PCT/US2012/59030 to VanLandingham et al. “Prophylactic and Post-Acute Use of Progesterone to Better Outcomes Associated with Concussion”
20. International PCT Patent Application No. PCT/US2012/59087 to VanLandingham et al. “Nasal Delivery Mechanism for Prophylactic and Post-Acute Use for Progesterone and/or Its Enantiomer for Use in Treatment of Mild Traumatic Brain Injuries”
21. International PCT Patent Application No. PCT/US2012/59083 to VanLandingham et al. “Prophylactic and Post-Acute Use of Progesterone in Conjunction with its Enantiomer for Use in Treatment of Mild Traumatic Brain Injuries”
22. Bosch, M. P.; Camps, F.; Coll, J.; Guerrero, T.; Tatsuoka, T.; Meinwald, J. J. Org. Chem. 1986, 51, 773
23. Tsunoda, T.; Suzuki, M.; Noyori, R. Tetrahedron Lett. 1980, 21, 1357
24. Yamauchi, Noriaki; Natsubori, Yoshiaki; Murae, Tatsushi Bulletin of the Chemical Society of Japan (2000), 73(11), 2513-2519
25. Das, Biswanath; Banerjee, Joydeep; Chowdhury, Nikhil; Majhi, Anjoy; Holla, Harish, Synlett (2006), (12), 1879-1882.
26. Liu, Hsing-Jang; Ly, Tai Wei; Tai, Chia-Liang; Wu, Jen-Dar; Liang, Jinn-Kwei; Guo, Jiunn-Cheh; Tseng, Nai-Wen; Shia, Kak-Shan. Tetrahedron (2003), 59(8), 1209-1226.
27. Tai, Chia-Liang; Ly, Tai Wei; Wu, Jen-Dar; Shia, Kak-Shan; Liu, Hsing-Jang. Synlett (2001), (2), 214-217.
28. Jiro Tsuji, Hideo Nagashima, and Hisao Nemoto (1990), “General Synthetic Method for the preparation of Methyl Ketones from Terminal Olefins: 2-Decanone”, Org. Synth.; Coll. Vol. 7: 137.
29. Basavaiah, D.; Rao, A. J.; Satyanarayana, T. Chem. Rev. 2003, 103, 811
30. Batt, Frederic and Fache, Fabienne, European Journal of Organic Chemistry, 2011(30), 6039-6055, S6039/1-S6039/46; 2011
31. Song, J. and Hollingsworth, R. I., Tetrahedron: Asymmetry, 12(3), 387-391; 2001

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

What is claimed is:

1. A method for preparing ent-progesterone comprising the step of reacting a compound of the formula: 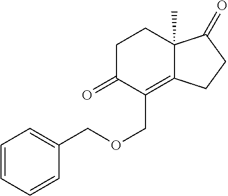 [SMILES: C[C@@]12CCC(=O)C(COCc3ccccc3)=C1CCC2=O]
to produce a compound of the formula:

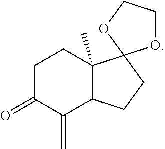 [SMILES: C=C1C(=O)CC[C@]2(C)C1CCC21OCCO1]

2. A method for preparing ent-progesterone comprising the step of reacting a compound of the formula: 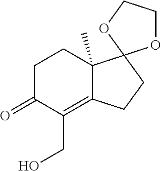 [SMILES: C[C@@]12CCC(=O)C(CO)=C1CCC21OCCO1]
to produce a compound of the formula:

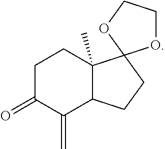 [SMILES: C=C1C(=O)CC[C@]2(C)C1CCC21OCCO1]

3. The method for preparing ent-progesterone according to claim 2, said method further comprising the step of subjecting a compound of the formula: 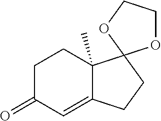
to a Baylis-Hillman reaction to produce a compound of the formula:

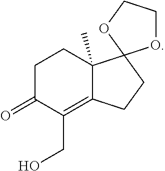 [SMILES: C[C@@]12CCC(=O)C(CO)=C1CCC21OCCO1]

4. A method for preparing ent-progesterone comprising the step of reacting a compound of the formula: 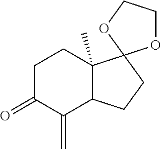 [SMILES: C=C1C(=O)CC[C@]2(C)C1CCC21OCCO1]
with a compound of the formula: 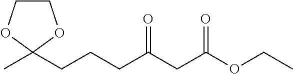 [SMILES: CCOC(=O)CC(=O)CCCC1(C)OCCO1]
to produce a compound of the formula:

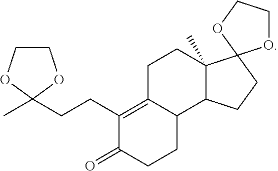 [SMILES: CC1(CCC2=C3CC[C@]4(C)C(CCC45OCCO5)C3CCC2=O)OCCO1]

5. The method for preparing ent-progesterone according to claim 4, wherein the compound of the formula 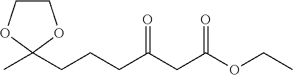 [SMILES: CCOC(=O)CC(=O)CCCC1(C)OCCO1]
is prepared by reacting a compound of the formula: 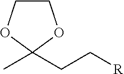
wherein R is any leaving group,
with a compound of the formula:

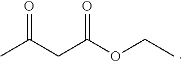

6. The method for preparing ent-progesterone according to claim 5, wherein R is —OTs, —OMs, —OTf, —Cl, —Br, or —I.

7. A method for preparing ent-progesterone comprising the step of reacting a compound of the formula: 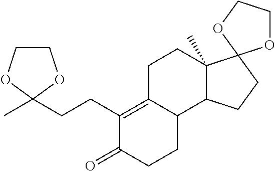 [SMILES: CC1(CCC2=C3CC[C@]4(C)C(CCC45OCCO5)C3CCC2=O)OCCO1]
to produce a compound of the formula:

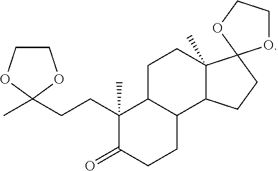 [SMILES: CC1(CC[C@]2(C)C(=O)CCC3C2CC[C@]2(C)C3CCC23OCCO3)OCCO1]

8. The method for preparing ent-progesterone according to claim 7, wherein the reaction step comprises a reductive silylation followed by de-silylation and methylation.

9. A method for preparing ent-progesterone comprising the step of reacting a compound of the formula: 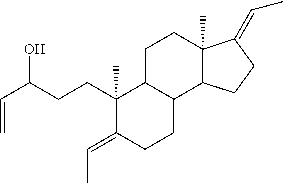 [SMILES: C=CC(O)CC[C@]1(C)/C(=C/C)CCC2C3CC/C(=C\C)[C@]3(C)CCC21]
to produce a compound of the formula:

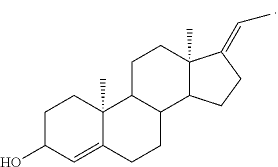 [SMILES: C/C=C1\CCC2C3CCC4=CC(O)CC[C@@]4(C)C3CC[C@@]12C]

10. A method for preparing ent-progesterone comprising the step of reacting a compound of the formula: 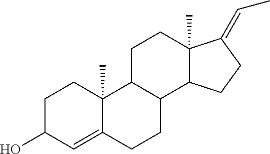 [SMILES: C/C=C1\CCC2C3CCC4=CC(O)CC[C@@]4(C)C3CC[C@@]12C]
to produce a compound of the formula:

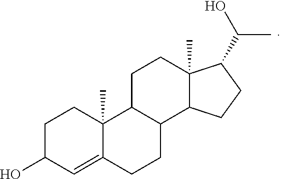 [SMILES: CC(O)[C@@H]1CCC2C3CCC4=CC(O)CC[C@@]4(C)C3CC[C@]21C]

11. A method for preparing ent-progesterone comprising the step of reacting a compound of the formula: 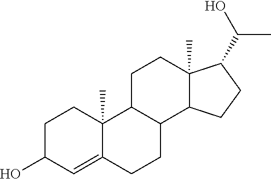 [SMILES: CC(O)[C@@H]1CCC2C3CCC4=CC(O)CC[C@@]4(C)C3CC[C@]21C]
to produce a compound of the formula:

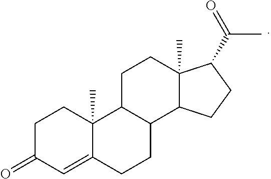 [SMILES: CC(=O)[C@@H]1CCC2C3CCC4=CC(=O)CC[C@@]4(C)C3CC[C@]21C]

12. A method for preparing ent-progesterone comprising the step of reacting a compound of the formula: 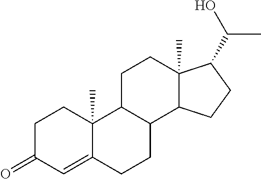 [SMILES: CC(O)[C@@H]1CCC2C3CCC4=CC(=O)CC[C@@]4(C)C3CC[C@]21C]
to produce a compound of the formula:

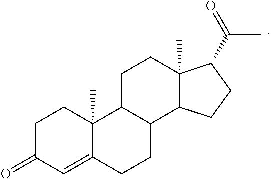 [SMILES: CC(=O)[C@@H]1CCC2C3CCC4=CC(=O)CC[C@@]4(C)C3CC[C@]21C]

13. A method for preparing ent-progesterone comprising the step of reacting a compound of the formula: 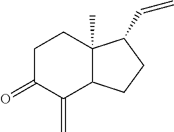
with a compound of the formula 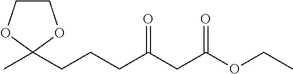 [SMILES: CCOC(=O)CC(=O)CCCC1(C)OCCO1]
to produce a compound of the formula

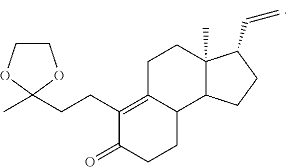 [SMILES: C=C[C@@H]1CCC2C3CCC(=O)C(CCC4(C)OCCO4)=C3CC[C@]21C]

14. The method for preparing ent-progesterone according to claim 10, wherein the compound of the formula 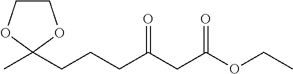 [SMILES: CCOC(=O)CC(=O)CCCC1(C)OCCO1]
is prepared by reacting a compound of the formula: 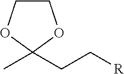
wherein R is any leaving group,
with a compound of the formula:

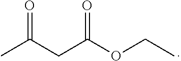

15. The method for preparing ent-progesterone according to claim 14, wherein R is —OTs, —OMs, —OTf, —Cl, —Br, or —I.

16. A method for preparing ent-progesterone comprising the step of reacting a compound of the formula: 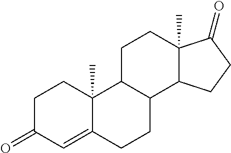 [SMILES: C[C@@]12CCC3C(CCC4=CC(=O)CC[C@]43C)C1CCC2=O]
to produce a compound of the formula

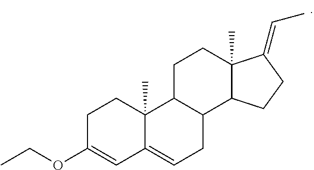 [SMILES: C/C=C1\CCC2C3CC=C4C=C(OCC)CC[C@@]4(C)C3CC[C@@]12C]

17. A method for preparing ent-progesterone comprising the step of reacting a compound of the formula: 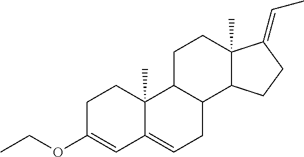 [SMILES: C/C=C1\CCC2C3CC=C4C=C(OCC)CC[C@@]4(C)C3CC[C@@]12C]
to produce a compound of the formula

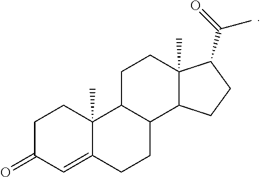 [SMILES: CC(=O)[C@@H]1CCC2C3CCC4=CC(=O)CC[C@@]4(C)C3CC[C@]21C]

18. A method for preparing ent-progesterone comprising the step of reacting a compound of the formula: 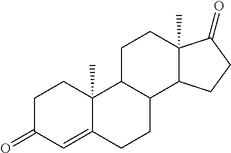 [SMILES: C[C@@]12CCC3C(CCC4=CC(=O)CC[C@]43C)C1CCC2=O]
to produce a compound of the formula

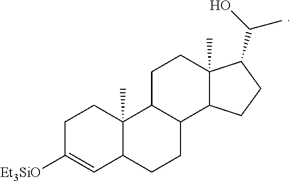

19. A method for preparing ent-progesterone comprising the step of reacting a compound of the formula: 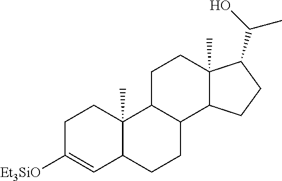
to produce a compound of the formula

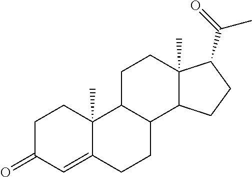 [SMILES: CC(=O)[C@@H]1CCC2C3CCC4=CC(=O)CC[C@@]4(C)C3CC[C@]21C]

20. A method for preparing ent-progesterone comprising the step of reacting a compound of the formula: 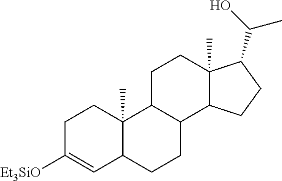
to produce a compound of the formula

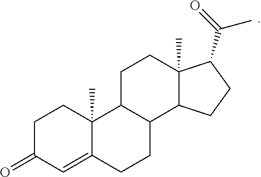 [SMILES: CC(=O)[C@@H]1CCC2C3CCC4=CC(=O)CC[C@@]4(C)C3CC[C@]21C]

21. A method for preparing ent-progesterone comprising the step of reacting an enone intermediate compound with triethylsilane and a catalyst to form a silyl enol ether.

22. The method for preparing ent-progesterone according to claim 1, wherein said method has fewer than 17 linear steps.

23. The method for preparing ent-progesterone according to claim 1, wherein said method has fewer than 15 linear steps.

24. The method for preparing ent-progesterone according to claim 1, wherein said method has fewer than 13 linear steps.

25. The method for preparing ent-progesterone according to claim 1, wherein said method has fewer than 12 linear steps.

26. The method for preparing ent-progesterone according to claim 2, wherein said method has fewer than 17 linear steps.

27. The method for preparing ent-progesterone according to claim 2, wherein said method has fewer than 15 linear steps.

28. The method for preparing ent-progesterone according to claim 2, wherein said method has fewer than 13 linear steps.

29. The method for preparing ent-progesterone according to claim 2, wherein said method has fewer than 12 linear steps.

30. The method for preparing ent-progesterone according to claim 3, wherein said method has fewer than 17 linear steps.

31. The method for preparing ent-progesterone according to claim 3, wherein said method has fewer than 15 linear steps.

32. The method for preparing ent-progesterone according to claim 3, wherein said method has fewer than 13 linear steps.

33. The method for preparing ent-progesterone according to claim 3, wherein said method has fewer than 12 linear steps.

34. The method for preparing ent-progesterone according to claim 4, wherein said method has fewer than 17 linear steps.

35. The method for preparing ent-progesterone according to claim 4, wherein said method has fewer than 15 linear steps.

36. The method for preparing ent-progesterone according to claim 4, wherein said method has fewer than 13 linear steps.

37. The method for preparing ent-progesterone according to claim 4, wherein said method has fewer than 12 linear steps.

38. The method for preparing ent-progesterone according to claim 5, wherein said method has fewer than 17 linear steps.

39. The method for preparing ent-progesterone according to claim 5, wherein said method has fewer than 15 linear steps.

40. The method for preparing ent-progesterone according to claim 5, wherein said method has fewer than 13 linear steps.

41. The method for preparing ent-progesterone according to claim 5, wherein said method has fewer than 12 linear steps.

42. The method for preparing ent-progesterone according to claim 6, wherein said method has fewer than 17 linear steps.

43. The method for preparing ent-progesterone according to claim 6, wherein said method has fewer than 15 linear steps.

44. The method for preparing ent-progesterone according to claim 6, wherein said method has fewer than 13 linear steps.

45. The method for preparing ent-progesterone according to claim 6, wherein said method has fewer than 12 linear steps.

46. The method for preparing ent-progesterone according to claim 7, wherein said method has fewer than 17 linear steps.

47. The method for preparing ent-progesterone according to claim 7, wherein said method has fewer than 15 linear steps.

48. The method for preparing ent-progesterone according to claim 7, wherein said method has fewer than 13 linear steps.

49. The method for preparing ent-progesterone according to claim 7, wherein said method has fewer than 12 linear steps.

50. The method for preparing ent-progesterone according to claim 8, wherein said method has fewer than 17 linear steps.

51. The method for preparing ent-progesterone according to claim 8, wherein said method has fewer than 15 linear steps.

52. The method for preparing ent-progesterone according to claim 8, wherein said method has fewer than 13 linear steps.

53. The method for preparing ent-progesterone according to claim 8, wherein said method has fewer than 12 linear steps.

54. The method for preparing ent-progesterone according to claim 9, wherein said method has fewer than 17 linear steps.

55. The method for preparing ent-progesterone according to claim 9, wherein said method has fewer than 15 linear steps.

56. The method for preparing ent-progesterone according to claim 9, wherein said method has fewer than 13 linear steps.

57. The method for preparing ent-progesterone according to claim 9, wherein said method has fewer than 12 linear steps.

58. The method for preparing ent-progesterone according to claim 10, wherein said method has fewer than 17 linear steps.

59. The method for preparing ent-progesterone according to claim 10, wherein said method has fewer than 15 linear steps.

60. The method for preparing ent-progesterone according to claim 10, wherein said method has fewer than 13 linear steps.

61. The method for preparing ent-progesterone according to claim 10, wherein said method has fewer than 12 linear steps.

62. The method for preparing ent-progesterone according to claim 11, wherein said method has fewer than 17 linear steps.

63. The method for preparing ent-progesterone according to claim 11, wherein said method has fewer than 15 linear steps.

64. The method for preparing ent-progesterone according to claim 11, wherein said method has fewer than 13 linear steps.

65. The method for preparing ent-progesterone according to claim 11, wherein said method has fewer than 12 linear steps.

66. The method for preparing ent-progesterone according to claim 12, wherein said method has fewer than 17 linear steps.

67. The method for preparing ent-progesterone according to claim 12, wherein said method has fewer than 15 linear steps.

68. The method for preparing ent-progesterone according to claim 12, wherein said method has fewer than 13 linear steps.

69. The method for preparing ent-progesterone according to claim 12, wherein said method has fewer than 12 linear steps.

70. The method for preparing ent-progesterone according to claim 13, wherein said method has fewer than 17 linear steps.

71. The method for preparing ent-progesterone according to claim 13, wherein said method has fewer than 15 linear steps.

72. The method for preparing ent-progesterone according to claim 13, wherein said method has fewer than 13 linear steps.

73. The method for preparing ent-progesterone according to claim 13, wherein said method has fewer than 12 linear steps.

74. The method for preparing ent-progesterone according to claim 14, wherein said method has fewer than 17 linear steps.

75. The method for preparing ent-progesterone according to claim 14, wherein said method has fewer than 15 linear steps.

76. The method for preparing ent-progesterone according to claim 14, wherein said method has fewer than 13 linear steps.

77. The method for preparing ent-progesterone according to claim 14, wherein said method has fewer than 12 linear steps.

78. The method for preparing ent-progesterone according to claim 15, wherein said method has fewer than 17 linear steps.

79. The method for preparing ent-progesterone according to claim 15, wherein said method has fewer than 15 linear steps.

80. The method for preparing ent-progesterone according to claim 15, wherein said method has fewer than 13 linear steps.

81. The method for preparing ent-progesterone according to claim 15, wherein said method has fewer than 12 linear steps.

82. The method for preparing ent-progesterone according to claim 16, wherein said method has fewer than 17 linear steps.

83. The method for preparing ent-progesterone according to claim 16, wherein said method has fewer than 15 linear steps.

84. The method for preparing ent-progesterone according to claim 16, wherein said method has fewer than 13 linear steps.

85. The method for preparing ent-progesterone according to claim 16, wherein said method has fewer than 12 linear steps.

86. The method for preparing ent-progesterone according to claim 17, wherein said method has fewer than 17 linear steps.

87. The method for preparing ent-progesterone according to claim 17, wherein said method has fewer than 15 linear steps.

88. The method for preparing ent-progesterone according to claim 17, wherein said method has fewer than 13 linear steps.

89. The method for preparing ent-progesterone according to claim 17, wherein said method has fewer than 12 linear steps.

90. The method for preparing ent-progesterone according to claim 18, wherein said method has fewer than 17 linear steps.

91. The method for preparing ent-progesterone according to claim 18, wherein said method has fewer than 15 linear steps.

92. The method for preparing ent-progesterone according to claim 18, wherein said method has fewer than 13 linear steps.

93. The method for preparing ent-progesterone according to claim 18, wherein said method has fewer than 12 linear steps.

94. The method for preparing ent-progesterone according to claim 19, wherein said method has fewer than 17 linear steps.

95. The method for preparing ent-progesterone according to claim 19, wherein said method has fewer than 15 linear steps.

96. The method for preparing ent-progesterone according to claim 19, wherein said method has fewer than 13 linear steps.

97. The method for preparing ent-progesterone according to claim 19, wherein said method has fewer than 12 linear steps.

98. The method for preparing ent-progesterone according to claim 20, wherein said method has fewer than 17 linear steps.

99. The method for preparing ent-progesterone according to claim 20, wherein said method has fewer than 15 linear steps.

100. The method for preparing ent-progesterone according to claim 20, wherein said method has fewer than 13 linear steps.

101. The method for preparing ent-progesterone according to claim 20, wherein said method has fewer than 12 linear steps.

102. The method for preparing ent-progesterone according to claim 21, wherein said method has fewer than 17 linear steps.

103. The method for preparing ent-progesterone according to claim 21, wherein said method has fewer than 15 linear steps.

104. The method for preparing ent-progesterone according to claim 21, wherein said method has fewer than 13 linear steps.

105. The method for preparing ent-progesterone according to claim 21, wherein said method has fewer than 12 linear steps.
